SEQUENCE LISTING (17 sequences)

SEQ ID NO: 1 - DNA, 413 bp, Homo sapiens
ttgtgaaatgtttaggaccactagacccggcgggcgcggcgacagcgacggagcgtcccacgcgcggcctggagtcagagtcacagtcaggggtgcgccggcgaccaatccaggggagcccacggaccggctccgcgctccaggtcggccgccccatgcgccgcgccccgcagtccccagggcctggctggctccacctgctgccagggaagaccctcggccggagagccaagtgctgctgggaggtggccgggcgtggccgggctgtgctgtgggaaccgctgggtgggggcgcgctgggttgcccacgctcctggcgcgctgccggcctggagacgccgccgccgccctcttgcaccccgagggcccgcacctcgaaactccgtggaattcccagcgaggaactggcggtg

SEQ ID NO: 2 - DNA, 23 bp, Artificial Sequence
  Sense primer
gttttatttgttgttagggaaga

SEQ ID NO: 3 - DNA, 18 bp, Artificial Sequence
  Antisense primer
ccacccccaattcctata

SEQ ID NO: 4 - DNA, 19 bp, Artificial Sequence
  First Probe
cgcgccaaaaacgtaaaca

SEQ ID NO: 5 - DNA, 19 bp, Artificial Sequence
  Second Probe
taaacaacccaacgcgccc

SEQ ID NO: 6 - DNA, 19 bp, Artificial Sequence
  Sense Primer
gaaccaaaacgctccccat

SEQ ID NO: 7 - DNA, 27 bp, Artificial Sequence
  Antisense Primer
ttatatgtcggttacgtgcgtttatat

SEQ ID NO: 8 - DNA, 22 bp, Artificial Sequence
  Probe
cccgtcgaaaacccgccgatta

SEQ ID NO: 9 - DNA, 19 bp, Artificial Sequence
  Sense Primer
cggaagcgttcgggtaaag

SEQ ID NO: 10 - DNA, 18 bp, Artificial Sequence
  Antisense Primer
aattccaccgccccaaac

SEQ ID NO: 11 - DNA, 29 bp, Artificial Sequence
  Probe
tttccgccaaatatcttttcttcttcgca

SEQ ID NO: 12 - DNA, 18 bp, Artificial Sequence
  Sense Primer
gcgttgaagtcggggttc

SEQ ID NO: 13 - DNA, 26 bp, Artificial Sequence
  Antisense Primer
aaacccgtacttcgctaactttaaac

SEQ ID NO: 14 - DNA, 24 bp, Artificial Sequence
  Probe
acaaacgcgaaccgaacgaaacca

SEQ ID NO: 15 - DNA, 27 bp, Artificial Sequence
  Sense Primer
ggtggaggtagttagggtttatttgta

SEQ ID NO: 16 - DNA, 31 bp, Artificial Sequence
  Antisense Primer
ccacaccacaaaatcacacttaacctcattt

SEQ ID NO: 17 - DNA, 21 bp, Artificial Sequence
  Probe
cacttttattcaactaatctc

(19) United States
(12) Patent Application Publication
(10) Pub. No.: US 2017/0183737 A1
(43) Pub. Date: Jun. 29, 2017

(54) METHOD FOR ESTIMATING A RISK FOR A SUBJECT SUFFERING FROM HEPATOCELLULAR CARCINOMA AND METHOD FOR THE PROGNOSIS OF HEPATOCELLULAR CARCINOMA

(71) Applicant: Industrial Technology Research Institute, Hsinchu (TW)

(72) Inventors: Chang-Yi LU, New Taipei City (TW); Chia-Jui YEN, Tainan City (TW); Shih-Ya CHEN, Taichung City (TW); Chia-Ju LIN, New Taipei City (TW); Yi-Chen LIU, Hsinchu City (TW); Pu-Yeh KAN, Keelung City (TW); Hui-Ling PENG, Guanxi Township (TW); Wan-Chi CHANG, New Taipei City (TW); Chao-Yun TSAO, Hsinchu City (TW)

(73) Assignee: Industrial Technology Research Institute, Hsinchu (TW)

(21) Appl. No.: 14/984,254

(22) Filed: Dec. 30, 2015

(30) Foreign Application Priority Data
Dec. 29, 2015 (TW) 104144178

(51) Int. Cl. C12Q 1/68

(52) U.S. Cl. CPC C12Q 1/6886; C12Q 2600/154; C12Q 2600/178; C12Q 2600/118; C12Q 2600/16

(57) ABSTRACT

The disclosure provides a method for estimating a risk for a subject suffering from hepatocellular carcinoma, including: (a) determining methylation levels of APC gene, COX2 gene, RASSF1A gene and micro RNA-203 gene in a sample of a subject, respectively; (b) calculating a predicted score according to the methylation levels of the APC gene, COX2 gene, RASSF1A gene and micro RNA-203 gene; and (c) estimating a risk level for the subject suffering from hepatocellular carcinoma according to the predicted score.

### CROSS REFERENCE TO RELATED APPLICATIONS

This application claims the priority benefit of Taiwan application serial No. 104144178, filed on Dec. 29, 2015. The entirety of the above-mentioned patent application is hereby incorporated by reference herein and made a part of this specification.

### INCORPORATION BY REFERENCE OF SEQUENCE LISTING

A sequence listing submitted as a text file via EFS-Web is incorporated herein by reference. The text file containing the sequence listing is named “0965-A24887-US_Seq_Listing.txt”; its date of creation is Jun. 2, 2016; and its size is 3,882 bytes.

### BACKGROUND

Technical Field

The technical field relates to the method for estimating the risk of a subject suffering from hepatocellular carcinoma and the method for prognosis of hepatocellular carcinoma.

Background

In general, abnormal DNA methylation can be observed in all of the cancer. DNA methylation is catalyzed by DNA methyltransferase via addition of a methyl group on the fifth carbon of cytosine. Instead, if DNA methylation occurs on the 5′ end of the gene or the CpG islands of the promoter region, transcription of the gene is often suppressed and thus resulting in non-activation of the gene. During the process of tumorigenesis, the phenomenon of abnormal DNA methylation is often involved in inhibition of DNA repair genes and tumor suppressor genes.

Due to abnormal DNA methylation usually occurs in early stage of cancers, it is very suitable as an index for a variety of cancers, such as classification of cancer, diagnosis, prognosis, risk assessment, response to chemotherapy and so on. Compared to other biomarkers, DNA methylation has its unique advantages, one of which is displaying its specificity between various tissues or different cancers. In addition, DNA methylation marker is a DNA marker and relative stable than RNA and protein. Specifically, in addition to be detected in tissue specimen, DNA methylation also can be detected in various body fluids, such as saliva, sputum, semen, gastrointestinal digestive, respiratory fluid, plasma, serum, urine, stool specimen and so on.

Present screen of liver cancer is proceeded by combining examinations of detecting fetoprotein (Alpha-Fetoprotein, AFP) index with abdominal ultrasound. However, both examinations of fetoprotein (AFP) and abdominal ultrasound have their limitations. According to statistics, about 70% to 80% of patients with liver cancer can be detected with increased fetoprotein index, but still about 20% of patients, even with late stage of liver cancer, cannot be detected with increased fetoprotein index.

For the diagnosis in early stage of liver cancer, the referential meaning of fetoprotein index is lower since one-third of small hepatocellular carcinoma (less than 3 cm) patients cannot be detected with increased fetoprotein index. Further, there are many other factors such as hepatitis, cirrhosis, pregnancy, and germ cell tumors that can cause increased fetoprotein index and affect the accuracy of diagnosis of liver cancer. Although the examination of ultrasound is no pain and no side effects, it requires highly trained physician to operate. Namely, the detection rate is relevant to the training and experience of the physician. In addition, ultrasound itself has some limitations, for example, some tumors growing in the blind angle of ultrasonic monitoring, unable to distinguish the nature of the tumor, and some invasive tumors or small tumors failed to be detected.

Until now, surgery is the only curative treatment for liver cancer. However, most hepatoma patients failed to proceed surgery to remove the tumor because it's difficult to sense the symptom at early stage of liver cancer and these patients are often diagnosed with accompanied liver dysfunction (more than 75% of patients with potential chronic liver disease), right and left lobes liver disease, or extrahepatic metastasis when identified with hepatoma. Therefore, the overall rate of resection in liver cancer is only 10% to 25%. If the tumor of hepatoma cannot be surgically removed, the prognosis would be poor and the median survival would be only a few months.

Therefore, it is urgent to develop new methods to detect liver cancer in order to improve the detection rate at early stage of liver cancer.

### SUMMARY

One embodiment of the present disclosure provides a method for evaluating the risk of liver cancer in a subject, comprising: (a) detecting methylation levels of APC gene, COX2 gene, RASSF1A gene and miR-203 gene respectively in a bio-sample from the subject; (b) calculating a predicted score A according to the methylation levels of APC gene, COX2 gene, RASSF1A gene and miR-203 gene; and (c) evaluating the risk level of liver cancer in the subject according to the predicted score A.

Another embodiment of the present disclosure provides a method for evaluating the risk of afflicting with hepatitis B virus-related liver cancer in a subject infected with hepatitis B virus, comprising: (a) detecting methylation levels of APC gene, COX2 gene, RASSF1A gene and miR-203 gene respectively in a bio-sample from the subject infected with hepatitis B virus; (b) calculating a predicted score B according to the methylation levels of APC gene, COX2 gene, RASSF1A gene and miR-203 gene; and (c) evaluating the risk level of afflicting with hepatitis B virus-related liver cancer in the subject infected with hepatitis B virus according to the predicted score B.

Another embodiment of the present disclosure provides a method for preparing the kit for APC gene, COX2 gene, RASSF1A gene and miR-203 gene for evaluating the risk of liver cancer in a subject.

Another embodiment of the present disclosure provides a method for preparing the kit for APC gene, COX2 gene, RASSF1A gene and miR-203 gene for evaluating the risk of afflicting with hepatitis B-related liver cancer in a subject infected with hepatitis B virus.

Another embodiment of the present disclosure provides a method for evaluating the prognosis of a subject afflicted with liver cancer, comprising: (a) detecting methylation levels of APC gene, COX2 gene, RASSF1A gene and miR-203 gene respectively in a bio-sample from the subject afflicted with liver cancer; (b) calculating a predicted score A according to the methylation levels of APC gene, COX2 gene, RASSF1A gene and miR-203 gene; and (c) evaluating the five-year survival probability of the subject afflicted with liver cancer according to the predicted score A.

Another embodiment of the present disclosure provides a method for evaluating a prognosis of a subject afflicted with liver cancer, comprising: (a) detecting methylation levels of APC gene, COX2 gene, RASSF1A gene and miR-203 gene respectively in a bio-sample from the subject afflicted with liver cancer; (b) calculating a predicted score according to the methylation levels of APC gene, COX2 gene, RASSF1A gene and miR-203 gene, and age, gender, AFP value, level of vascular invasion, tumor size, clinical stage, suffering from hepatitis virus or not and suffering from cirrhosis or not; and (c) evaluating the survival probability in 5 years of the subject afflicted with liver cancer according to the predicted score.

Another embodiment of the present disclosure provides a kit for detecting methylation level of miR-203 gene, comprising: a primer-pair including a sense primer and an antisense primer and a first probe and/or a second probe, in which the sense primer has a sequence of at least 85% sequence similarity to SEQ IDNO: 2, the antisense primer has a sequence of at least 85% sequence similarity to SEQ IDNO: 3, the first probe has a sequence of at least 85% sequence similarity to SEQ IDNO: 4, and the second probe has a sequence of at least 85% sequence similarity to SEQ IDNO: 5.

Another embodiment of the present disclosure provides a kit for evaluating a risk of liver cancer in a subject and/or evaluating a prognosis of a subject afflicted with liver cancer, comprising: a primer-pair and a probe for detecting methylation level of miR-203 gene, a primer-pair and a probe for detecting methylation level of APC gene, a primer-pair and a probe for detecting methylation level of COX2 gene, and a primer-pair and a probe for detecting methylation level of RASSF1A gene.

### BRIEF DESCRIPTION OF THE DRAWINGS

The present disclosure can be more fully understood by reading the subsequent detailed description and exemplary embodiments with references to the accompanying drawings so as to be easily realized by a person having ordinary knowledge in the art, wherein:

FIG. 1 indicates the methylation level of APC gene in different diseases groupings;

FIG. 2 indicates the methylation level of COX2 gene in different diseases groupings;

FIG. 3 indicates the methylation level of micro RNA-203 gene in different diseases groupings;

FIG. 4 indicates the methylation level of RASSF1A gene in different diseases groupings;

FIG. 5 indicates the analysis result of live cancer grouping via performing the univariate ln(APC) by logistic regression and receiver operating characteristic curve (ROC) analysis;

FIG. 6 indicates the analysis result of live cancer grouping via performing the univariate ln(COX2) by logistic regression and receiver operating characteristic curve (ROC) analysis;

FIG. 7 indicates the analysis result of live cancer grouping via performing the univariate ln(miR-203) by logistic regression and receiver operating characteristic curve (ROC) analysis;

FIG. 8 indicates the analysis result of live cancer grouping via performing the univariate ln(RASSF1A) by logistic regression and receiver operating characteristic curve (ROC) analysis;

FIG. 9 indicates the analysis result of live cancer grouping via performing four variates ln(APC), ln(COX2), ln(miR-203) and ln(RASSF1A) by logistic regression and receiver operating characteristic curve (ROC) analysis;

FIG. 10 indicates the analysis result of live cancer grouping via performing four variates ln(APC), ln(COX2), ln(miR-203) and ln(RASSF1A) by cross validation and receiver operating characteristic curve (ROC) analysis;

FIG. 11 indicates the analysis result of live cancer grouping via performing the univariate AFP by logistic regression and receiver operating characteristic curve (ROC) analysis;

FIG. 12 indicates the analysis result of hepatitis B-related live cancer grouping via performing the univariate ln(APC) by logistic regression and receiver operating characteristic curve (ROC) analysis;

FIG. 13 indicates the analysis result of hepatitis B-related live cancer grouping via performing the univariate ln(COX2) by logistic regression and receiver operating characteristic curve (ROC) analysis;

FIG. 14 indicates the analysis result of hepatitis B-related live cancer grouping via performing the univariate ln(miR-203) by logistic regression and receiver operating characteristic curve (ROC) analysis;

FIG. 15 indicates the analysis result of hepatitis B-related live cancer grouping via performing the univariate ln(RASSF1A) by logistic regression and receiver operating characteristic curve (ROC) analysis;

FIG. 16 indicates the analysis result of hepatitis B-related live cancer grouping via performing four variates ln(APC), ln(COX2), ln(miR-203) and ln(RASSF1A) by logistic regression and receiver operating characteristic curve (ROC) analysis;

FIG. 17 indicates the analysis result of hepatitis B-related live cancer grouping via performing four variates ln(APC), ln(COX2), ln(miR-203) and ln(RASSF1A) by cross validation and receiver operating characteristic curve (ROC) analysis;

FIG. 18 indicates the analysis result of hepatitis B-related live cancer grouping via performing the univariate AFP by logistic regression and receiver operating characteristic curve (ROC) analysis;

FIG. 19 indicates the univariate analysis result of the 5-year survival probability for the predicted score A higher than 0.45 or not;

FIG. 20 indicates the estimated 5-year survival function, which is performed by using Cox proportional hazards model to calculate the prognosis score, using Breslow to calculate the basic survival function, and grouping via the predicted score A higher than 0.45 or not to adjust the median of the prognosis score;

FIG. 21 indicates the estimated 5-year survival function in different sub-groupings, which is performed by using Cox proportional hazards model to calculate the prognosis score, using Breslow to calculate the basic survival function, and grouping via the predicted score A (higher than 0.45 or not) and the AFP value (higher than 20 or not) to adjust the median of the prognosis score.

### DETAILED DESCRIPTION

In the following description, one embodiment of the present disclosure provides a method for evaluating the risk of suffering liver cancer in a subject. It is not particularly limited the type of liver cancer suitable to be assessed by the method for evaluating the risk of suffering liver cancer in a subject. In one embodiment, the type of liver cancer suitable to be assessed by the method for evaluation includes hepatitis B-related liver cancer.

The above-mentioned method for evaluating the risk of suffering liver cancer in a subject may include, but not limited to the following steps. The first step is to detect the methylation levels of APC gene, COX2 gene, RASSF1A gene and miR-203 gene respectively in a bio-sample from the subject. The above-mentioned subject may include, but not limited thereto a mammal, for example, human, ape, monkey, cat, dog, rabbit, guinea pig, rat or mouse. In one embodiment, the above-mentioned subject can be human. Further, the above-mentioned bio-sample may include, but not limited thereto, blood, plasma, serum, liver tissue, saliva, sputum, semen, intestinal digestive, respiratory lavage, feces and so on. In one embodiment, the above-mentioned bio-sample can be plasma or serum.

It is not particularly limited the methylation sites to be detected in APC gene, COX2 gene, RASSF1A gene, and miR-203 gene. In one embodiment, methylation of miR-203 gene can be detected within the sequence between position 104,522,452 base pair (bp) and 104,522,886 bp of chromosome 14 (based on NCBI Homo sapiens Annotation Release 107) (SEQ ID NO: 1), including further confirming the methylation level or status of the CpG dinucleotides between position 104,522,554 bp and 104,522,557 bp, and/or between position 104,522,570 bp and 104,522,571 bp, and/or between position 104,522,579 bp and 104,522,582 bp.

Moreover, the method suitable for detection the methylation status of APC gene, COX2 gene, RASSF1A gene and miR-203 gene may include, but not limited thereto, quantitative methylation-specific polymerase chain reaction (quantitative methylation-specific PCR, qMSP), combined bisulfite restriction analysis (COBRA), Bisulfite Sequencing, Pyrosequencing, Next Generation sequencing (NGS), DNA Methylation Array Chip Analysis and so on. In one embodiment, the methylation status is detected by the method of quantitative methylation-specific PCR.

In a particular embodiment, the methylation status is detected by the method of quantitative methylation-specific PCR, and the methylation sites to be detected in miR-203 gene can refer to the above-mentioned methylation sites, and no more repeat is needed here.

In the above-mentioned particular embodiment, the methylation level or status of miR-203 gene is detected by combining a primer-pair, a first probe and/or a second probe. The primer-pair includes a sense primer and an antisense primer, in which the sense primer has a sequence of at least 85% sequence similarity to SEQ IDNO: 2, and the antisense primer has a sequence of at least 85% sequence similarity to SEQ IDNO: 3. The first probe has a sequence of at least 85% sequence similarity to SEQ IDNO: 4 and the second probe has a sequence of at least 85% sequence similarity to SEQ IDNO: 5. In one embodiment, the methylation status of miR-203 gene is detected by combining a primer-pair, a first probe and/or a second probe, in which the primer-pair includes a sense primer and an antisense primer, the sense primer has a sequence as set forth in SEQ IDNO: 2, and the antisense primer has a sequence as set forth in SEQ IDNO: 3, the first probe has a sequence as set forth in SEQ IDNO: 4, and the second probe has a sequence as set forth in SEQ IDNO: 5.

The predicted score A is based on the methylation levels of APC gene, COX2 gene, RASSF1A gene and miR-203 gene, and analyzed by the method including, but not limited thereto, logistic regression analysis, discriminant function analysis, ridge regression analysis and so on. In one embodiment, the predicted score A is based on the methylation levels of APC gene, COX2 gene, RASSF1A gene and miR-203 gene, and analyzed by the method of logistic regression analysis.

In one embodiment, the predicted score A is based on the methylation levels of APC gene, COX2 gene, RASSF1A gene and miR-203 gene and calculated by the following formula:

Predicted score A=exp(predicted value A)/(1+exp(predicted value A)), in which the predicted value A=X₁×X₂×ln(APC)+X₃×ln(COX2)+X₄×ln(miR-203)+X₅×ln(RASSF1A), X₁ ranges from 1.6148 to 2.8618, X₂ ranges from 0.0237 to 0.1559, X₃ ranges from 0.1169 to 0.2581, X₄ ranges from 0.0058 to 0.1344, and X₅ ranges from 0.0436 to 0.1758. In addition, ln(APC) represents a hyperbolic logarithm of the methylation level of APC gene, and the methylation level of APC gene is calculated from the following formula: 2̂(Ct(β-actin)−Ct(APC))×1000, ln(COX2) represents a hyperbolic logarithm of the methylation level of COX2 gene, and the methylation level of COX2 gene is calculated from the following formula: 2̂(Ct(β-actin)−Ct(COX2))×1000, ln(miR-203) represents a hyperbolic logarithm of the methylation level of miR-203 gene, and the methylation level of miR-203 gene is calculated from the following formula: 2̂(Ct(β-actin)−Ct(miR-203))×1000, and ln(RASSF1A) represents a hyperbolic logarithm of the methylation level of RASSF1A gene, and the methylation level of RASSF1A gene is calculated from the following formula: 2̂(Ct(β-actin)−Ct(RASSF1A))×1000.

In one particular embodiment of above-mentioned formula, X₁ is 2.238, X₂ is 0.0898, X₃ is 0.1875, X₄ is 0.0701, and X₅ is 0.1097.

After calculating a predicted score A according to the methylation levels of APC gene, COX2 gene, RASSF1A gene and miR-203 gene, evaluating a risk level of liver cancer in the subject according to the predicted score A is executed. If the predicted score A is higher relative to a pre-confirmed reference value, it indicates that the subject has the risk of afflicting with liver cancer.

In one embodiment, the pre-confirmed reference value is determined by comparing the methylation levels of APC gene, COX2 gene, RASSF1A gene and miR-203 gene in one group of subjects known to non-live cancer with another group known to live cancer and obtaining a cutoff value according to the receiver operating characteristic (ROC) curve. In one particular embodiment, if the pre-confirmed reference value is 0.45 and the predicted score A is higher than 0.45, the subject has the risk of afflicting with liver cancer.

A method of preparing a kit for APC gene, COX2 gene, RASSF1A gene and miR-203 gene is also provided in another embodiment of the present disclosure, in which the kit is used for the method of evaluating the risk of afflicting with liver cancer in a subject.

A method for evaluating a risk of afflicting with hepatitis B-related liver cancer in a subject infected with hepatitis B virus is also provided in another embodiment of the present disclosure and comprises the following steps, but not limited thereto. First, detecting the methylation levels of APC gene, COX2 gene, RASSF1A gene and miR-203 gene respectively in a bio-sample from the subject infected with hepatitis B virus is executed.

Regarding the subjects, samples, the methylation sites of miR-203 gene, the methods suitable for detecting methylation of gene, as well as the primer pair and probes for detecting methylation of miR-203 gene, are described as above-mentioned corresponding paragraphs, and it is no longer repeat them here.

Then, a predicted score B is calculated according to the methylation levels of APC gene, COX2 gene, RASSF1A gene and miR-203 gene. The predicted score B is based on the methylation levels of APC gene, COX2 gene, RASSF1A gene and miR-203 gene, and analyzed by the method including, but not limited thereto, logistic regression analysis, discriminant function analysis, ridge regression analysis and so on. In one embodiment, the predicted score B is based on the methylation levels of APC gene, COX2 gene, RASSF1A gene and miR-203 gene, and analyzed by the method of logistic regression analysis.

In one embodiment, the predicted score B is based on the methylation levels of APC gene, COX2 gene, RASSF1A gene and miR-203 gene and calculated by the following formula:

Predicted score B=exp(predicted value B)/(1+exp(predicted value B)), in which the predicted value B=Y₁+Y₂×ln(APC)+Y₃×ln(COX2)+Y₄×ln(miR-203)+Y₅×ln(RASSF1A), Y₁ ranges from 1.7 to 3.34, Y₂ ranges from 0.045 to 0.213, Y₃ ranges from 0.142 to 0.32, Y₄ ranges from 0.028 to 0.193, and Y₅ ranges from 0.038 to 0.224. In addition, ln(APC) represents a hyperbolic logarithm of the methylation level of APC gene, and the methylation level of APC gene is calculated from the following formula: 2̂(Ct(β-actin)−Ct(APC))×1000, ln(COX2) represents a hyperbolic logarithm of the methylation level of COX2 gene, and the methylation level of COX2 gene is calculated from the following formula: 2̂(Ct(β-actin)−Ct(COX2))×1000, ln(miR-203) represents a hyperbolic logarithm of the methylation level of miR-203 gene, and the methylation level of miR-203 gene is calculated from the following formula: 2̂(Ct(β-actin)−Ct(miR-203))×1000, and ln(RASSF1A) represents a hyperbolic logarithm of the methylation level of RASSF1A gene, and the methylation level of RASSF1A gene is calculated from the following formula: 2̂(Ct(β-actin)−Ct(RASSF1A))×1000.

In one particular embodiment of above-mentioned formula, Y₁ is 2.447, Y₂ is 0.127, Y₃ is 0.226, Y₄ is 0.1091, and Y₅ is 0.1288.

After calculating a predicted score B according to the methylation levels of APC gene, COX2 gene, RASSF1A gene and miR-203 gene, evaluating a risk level of afflicting with hepatitis B-related liver cancer in the subject infected with hepatitis B virus according to the predicted score B is executed. If the predicted score B is higher relative to a pre-confirmed reference value, it indicates that the subject infected with hepatitis B virus has the risk of afflicting with hepatitis B virus-related liver cancer.

In one embodiment, the pre-confirmed reference value is determined by comparing the methylation levels of APC gene, COX2 gene, RASSF1A gene and miR-203 gene in one group of subjects known to non-hepatitis B virus related live cancer with another group known to hepatitis B virus related live cancer and obtaining a cutoff value according to the receiver operating characteristic (ROC) curve. In one particular embodiment, if the pre-confirmed reference value is 0.4 and the predicted score B is higher than 0.4, the subject has the risk of afflicting with hepatitis B virus-related liver cancer.

A method of preparing a kit for APC gene, COX2 gene, RASSF1A gene and miR-203 gene is also provided in another embodiment of the present disclosure, in which the kit is utilized for the method of evaluating the risk of afflicting with hepatitis B-related liver cancer in a subject infected with hepatitis B virus.

A method for evaluating the prognosis of a subject afflicted with liver cancer is also provided in another embodiment of the present disclosure. The method for evaluating the prognosis of a subject afflicted with liver cancer is not limited. In one embodiment, the subject afflicted with liver cancer assessed the prognosis by the above-mentioned method may include the patients of hepatitis B-related liver cancer and the patients of hepatitis C-related liver cancer.

A method for evaluating the prognosis of a subject afflicted with liver cancer comprises the following steps, but not limited thereto. First, detecting the methylation levels of APC gene, COX2 gene, RASSF1A gene and miR-203 gene respectively in a bio-sample from the subject afflicted with liver cancer is executed. Regarding the subjects, samples, the methylation sites of miR-203 gene, the methods suitable for detecting methylation of gene, as well as the primer pair and probes for detecting methylation of miR-203 gene, are described as above-mentioned corresponding paragraphs, and it is no longer repeat them here.

Then, a predicted score A is calculated according to the methylation levels of APC gene, COX2 gene, RASSF1A gene and miR-203 gene, and the 5-year survival probability of the subject afflicted with liver cancer is evaluated according to the predicted score A.

The predicted score A is based on the methylation levels of APC gene, COX2 gene, RASSF1A gene and miR-203 gene and calculated by the following formula:

Predicted score A=exp(predicted value A)/(1+exp(predicted value A)), in which the predicted value A=X₁+X₂×ln(APC)+X₃×ln(COX2)+X₄×ln(miR-203)+X₅×ln(RASSF1A), X₁ ranges from 1.6148 to 2.8618, X₂ ranges from 0.0237 to 0.1559, X₃ ranges from 0.1169 to 0.2581, X₄ ranges from 0.0058 to 0.1344, and X₅ ranges from 0.0436 to 0.1758. In addition, ln(APC) represents a hyperbolic logarithm of the methylation level of APC gene, and the methylation level of APC gene is calculated from the following formula: 2̂(Ct((β-actin)−Ct(APC))×1000, ln(COX2) represents a hyperbolic logarithm of the methylation level of COX2 gene, and the methylation level of COX2 gene is calculated from the following formula: 2̂(Ct(β-actin)−Ct(COX2))×1000, ln(miR-203) represents a hyperbolic logarithm of the methylation level of miR-203 gene, and the methylation level of miR-203 gene is calculated from the following formula: 2̂(Ct(β-actin)−Ct(miR-203))×1000, and ln(RASSF1A) represents a hyperbolic logarithm of the methylation level of RASSF1A gene, and the methylation level of RASSF1A gene is calculated from the following formula: 2̂(Ct(β-actin)−Ct(RASSF1A))×1000.

In one embodiment, if the predicted score A is higher than a pre-confirmed reference value, it indicates that the five-year survival probability is about 20% to 30%. But if the predicted score A is lower than or equal to a pre-confirmed reference value, it indicates that the five-year survival probability is about 60% to 70%.

The pre-confirmed reference value is determined by comparing the methylation levels of APC gene, COX2 gene, RASSF1A gene and miR-203 gene in one group of subjects known to non-live cancer with another group known to live cancer, and obtains a cutoff value according to the receiver operating characteristic (ROC) curve. The pre-confirmed reference value is about 0.4 to 0.5, but not limited thereto. In one embodiment, if the pre-confirmed reference value is 0.45 and the predicted score A higher than 0.45, the five-year survival probability is about 26.53%. But if the predicted score A is lower than or equal to 0.45, the five-year survival probability is about 69.63%.

A method for evaluating the prognosis of a subject afflicted with liver cancer is also provided in another embodiment of the present disclosure. The method suitable for evaluating the prognosis of a subject afflicted with liver cancer is not limited. In one embodiment, the subject afflicted with liver cancer assessed the prognosis by the above-mentioned method may include the patients of hepatitis B-related liver cancer and the patients of hepatitis C-related liver cancer.

A method for evaluating the prognosis of a subject afflicted with liver cancer comprises the following steps, but not limited thereto. First, detecting the methylation levels of APC gene, COX2 gene, RASSF1A gene and miR-203 gene respectively in a bio-sample from the subject afflicted with liver cancer is executed. Regarding the subjects, samples, the methylation sites of miR-203 gene, the methods suitable for detecting methylation of gene, as well as the primer pair and probes for detecting methylation of miR-203 gene, such as described in above-mentioned corresponding paragraphs, it is no longer repeat them here.

Then, a predicted score is calculated by multivariate survival analysis according to the methylation levels of APC gene, COX2 gene, RASSF1A gene and miR-203 gene, and age, gender, AFP value, level of vascular invasion, tumor size, clinical stage, suffering from hepatitis virus or not and suffering from cirrhosis or not.

In one embodiment, evaluating the prognosis and the survival probability may comprise, but not limited thereto the following steps: (i) calculating a predicted score A according to the methylation levels of APC gene, COX2 gene, RASSF1A gene and miR-203 gene is executed; and (ii) calculating a predicted score is by combining the predicted score A with age, gender, AFP value, level of vascular invasion, tumor size, clinical stage, suffering from hepatitis virus or not and suffering from cirrhosis or not.

In above-mentioned step (i), the predicted score A is based on the methylation levels of APC gene, COX2 gene, RASSF1A gene and miR-203 gene and analyzed by the method including, but not limited thereto, logistic regression analysis, discriminant function analysis, ridge regression analysis and so on. In one embodiment, the predicted score A is based on the methylation levels of APC gene, COX2 gene, RASSF1A gene and miR-203 gene, and analyzed by the method of logistic regression analysis.

In one embodiment of the above-mentioned step (i), the predicted score A is based on the methylation levels of APC gene, COX2 gene, RASSF1A gene and miR-203 gene and calculated by the following formula:

Predicted score A=exp(predicted value A)/(1+exp(predicted value A)), in which the predicted value A=X₁+X₂×ln(APC)+X₃×ln(COX2)+X₄×ln(miR-203)+X₅×ln(RASSF1A), X₁ ranges from 1.6148 to 2.8618, X₂ ranges from 0.0237 to 0.1559, X₃ ranges from 0.1169 to 0.2581, X₄ ranges from 0.0058 to 0.1344, and X₅ ranges from 0.0436 to 0.1758. In addition, ln(APC) represents a hyperbolic logarithm of the methylation level of APC gene, and the methylation level of APC gene is calculated from the following formula: 2̂(Ct(β-actin)−Ct(APC))×1000, ln(COX2) represents a hyperbolic logarithm of the methylation level of COX2 gene, and the methylation level of COX2 gene is calculated from the following formula: 2̂(Ct(β-actin)−Ct(COX2))×1000, ln(miR-203) represents a hyperbolic logarithm of the methylation level of miR-203 gene, and the methylation level of miR-203 gene is calculated from the following formula: 2̂(Ct(β-actin)−Ct(miR-203))×1000, and ln(RASSF1A) represents a hyperbolic logarithm of the methylation level of RASSF1A gene, and the methylation level of RASSF1A gene is calculated from the following formula: 2̂(Ct(β-actin)−Ct(RASSF1A))×1000.

Further, in one particular embodiment of above-mentioned formula, X₁ is 2.238, X₂ is 0.0898, X₃ is 0.1875, X₄ is 0.0701, and X₅ is 0.1097.

In above-mentioned step (ii), the predicted score is calculated by multivariate survival analysis according to age, gender, AFP value, level of vascular invasion, tumor size, clinical stage, suffering from hepatitis virus or not, suffering from cirrhosis or not and the predicted score A higher relative to a pre-confirmed reference value or not.

In one embodiment of the above-mentioned step (ii), the predicted score is based on age, gender, AFP value, level of vascular invasion, tumor size, clinical stage, suffering from hepatitis virus or not, suffering from cirrhosis or not and the predicted score A higher relative to a pre-confirmed reference value or not, and calculated by the following formula:

Predicted score=B₁×(age)+B₂×(gender)+B₃×(AFP value higher than 20 or not)+B₄×(level of vascular invasion)+B₅×(tumor size higher than 5 cm or not)+B₆×(clinical stage)+B₇×(suffering from cirrhosis or not)+Bₛ×(predicted score A higher relative to a pre-confirmed reference value or not), in which B₁ ranges from −0.0224 to 0.0426, B₂ ranges from −0.8233 to 0.7836, B₃ ranges from 0.1798 to 1.3902, B₄ ranges from −0.1089 to 1.0898, B₅ ranges from −0.9560 to 0.4118, B₆ ranges from 0.8525 to 2.202, B₇ ranges from −1.9221 to −0.2812, and B₈ ranges from 0.3534 to 2.2217. In addition, age substitutes actual age, gender substitutes 1 for men and 0 for women, AFP value higher than 20 or not substitutes 1 for yes and 0 for no, level of vascular invasion substitutes 1 for yes and 0 for no, tumor size higher than 5 cm or not substitutes I for yes and 0 for no, clinical stage substitutes 1 for III/IV and 0 for I/II, suffering from cirrhosis or not substitutes 1 for yes and 0 for no, and predicted score A higher than pre-confirmed reference value or not substitutes 1 for yes and 0 for no.

Further, the pre-confirmed reference value is determined by comparing the methylation levels of APC gene, COX2 gene, RASSF1A gene and miR-203 gene in one group of subjects known to non-live cancer with another group known to live cancer and obtaining a cutoff value according to the receiver operating characteristic (ROC) curve. The pre-confirmed reference value is about 0.4 to 0.5, but not limited thereto. In one embodiment, the pre-confirmed reference value is 0.45.

After calculating the predicted score, the survival probability in the estimated survival time t (year) is calculated according to the predicted score. In one embodiment, survival probability in estimated survival time t (year) is calculated by the following formula: survival probability in estimated survival time t(year)=S0(t)exp(predicted score), in which S0(t) represents survival probability in t year.

In one particular embodiment, if the predicted score of the subject afflicted with liver cancer is less than 0.45, the 5-year survival probability is about 69.48%. But if the predicted score A is higher than or equal to 0.45, the 5-year survival probability is about 34.19%.

Based on the predicted score A and AFP value, and adjusted by median of composite value in other variables, ANCOVA (analysis of covariance) survival function is estimated and adjusted by the method of Breslow to illustrate the difference among four groupings of combination of predicted score A and AFP value.

When the subject suffering from liver cancer is detected with AFP value of less than or equal to 20 (ng/ml) and prediction score A of less than or equal to 0.45, the five-year survival probability is 69.48%. When the subject suffering from liver cancer is detected with AFP value of greater than 20 (ng/ml) and prediction score A of less than or equal to 0.45, the five-year survival probability is 48.61%. When the subject suffering from liver cancer is detected with AFP value of less than or equal to 20 (ng/ml) and prediction score A of greater than 0.45, the five-year survival probability is 34.19%. When the subject suffering from liver cancer is detected with AFP value of greater than 20 (ng/ml) and prediction score A of greater than 0.45, the five-year survival probability is remaining 11.64%.

In another embodiment of the present disclosure, a kit for detecting methylation level of miR-203 gene is provided to comprise, but not limited thereto a primer-pair including a sense primer and an antisense primer, and a first probe and/or a second probe.

In one embodiment, the sense primer has a sequence of at least 85% sequence similarity to SEQ IDNO: 2, and the antisense primer has a sequence of at least 85% sequence similarity to SEQ IDNO: 3. In addition, the first probe has a sequence of at least 85% sequence similarity to SEQ IDNO: 4, and the second probe has a sequence of at least 85% sequence similarity to SEQ IDNO: 5.

In one particular embodiment, a kit for detecting methylation level of miR-203 gene may comprise a primer-pair including a sense primer and an antisense primer, and a first probe and/or a second probe, in which the sense primer has a sequence as set forth in SEQ IDNO: 2, the antisense primer has a sequence as set forth in SEQ IDNO: 3, the first probe has a sequence as set forth in SEQ IDNO: 4, and the second probe has a sequence as set forth in SEQ IDNO: 5.

In another embodiment of the present disclosure, a kit for evaluating a risk of liver cancer in a subject and/or evaluating a prognosis of a subject afflicted with liver cancer is provided to comprise, but not limited thereto, a primer-pair and a probe for detecting methylation level of miR-203 gene, a primer-pair and a probe for detecting methylation level of APC gene, a primer-pair and a probe for detecting methylation level of COX2 gene, and a primer-pair and a probe for detecting methylation level of RASSF1A gene.

In one embodiment, a kit for detecting methylation level of miR-203 gene is provided to comprise a primer-pair and a first probe and/or a second probe, in which the primer-pair may include a sense primer and an antisense primer, and the probe may include a first probe and/or a second probe. Further, the sense primer has a sequence of at least 85% sequence similarity to SEQ IDNO: 2, and the antisense primer has a sequence of at least 85% sequence similarity to SEQ IDNO: 3. In addition, the first probe has a sequence of at least 85% sequence similarity to SEQ IDNO: 4, and the second probe has a sequence of at least 85% sequence similarity to SEQ IDNO: 5. In one embodiment, the above-mentioned kit is suitable for quantitative methylation-specific polymerase chain reaction, but not limited thereto.

### EXAMPLES

A. Detection of Gene Methylation

(1) Clinical Plasma Sample

357 cases of Clinical plasma samples are received from National Cheng Kung University Hospital, in which 50 cases of healthy, 47 cases of hepatitis (including 21 cases of hepatitis B and 26 cases of hepatitis C), 57 cases of hepatitis with cirrhosis (including 32 cases of hepatitis B and 25 cases of hepatitis C), and 203 cases of liver cancer (including 81 cases of hepatitis B, 30 cases of hepatitis C, 42 cases of hepatitis B with cirrhosis, and 50 cases of hepatitis C with cirrhosis). This clinical study is reviewed and approved by the Institutional Review Board (IRB) of National Cheng Kung University Hospital.

(2) Extraction of DNA

Extraction of DNA is executed with QIAGEN Q1Aamp DNA Blood Mini Kit according to the procedure recommended by the supplier, in which 800 μl of plasma sample is utilized for extraction of DNA, and the extracted DNA concentration is measured by real-time quantitative polymerase chain reaction (Q-PCR).

(3) Treatment with Sodium Bisulfite

EZ DNA methylation kit (Zymo Research) is utilized to treat the clinical sample DNA, including performing treatment with sodium bisulfite, and the treatment process is according to the procedure recommended by the supplier.

(4) Real-Time Quantitative Methylation Analysis

After conversed by above-mentioned sodium bisulfite, DNA is detected by real-time quantitative methylation-specific PCR (qMSP). Each reaction consists of 1× KAPA PROBE FAST Master Mix (KAPA), 0.5 μM sense primer and 0.5 μM antisense primer, and 0.25 μM probe with a total volume of 20 μl. Amplification is performed with StepOnePlus real-time PCR system (Thermo Fisher Scientific Inc.) according to the following thermal cycling conditions: 95° C. for 3 min, and then 95° C. for 3 seconds, 60-68° C. for 20 seconds, and 72° C. for 10 seconds with 55 cycles. Next, the methylation level or status is determined by the difference of Ct value between β-actin gene and target gene, and calculated by the following formula: 2 [Ct(β-actin)−Ct(target gene)]×1000.

The primer-pair and probe utilized for detecting methylation level of APC gene, COX2 gene, RASSF1A gene and miR-203 gene are illustrated as follows:

TABLE 1

| Target | Primer/ | |
|---|---|---|
| gene | Probe | Sequence |
| miRNA-203 | Sense | gTTTTATTTgTTgTTAgggAAgA |
| | primer | (SEQ ID No. 2) |
| | Antisense | CCACCCCCAATTCCTATA |
| | primer | (SEQ ID No. 3) |
| | Probe 1 | CgCgCCAAAAACgTAAACA |
| | | (SEQ ID No. 4) |
| | Probe 2 | TAAACAACCCAACgCgCCC |
| | | (SEQ ID No. 5) |
| APC | Sense | GAACCAAAACGCTCCCCAT |
| | primer | (SEQ ID No. 6) |
| | Antisense | TTATATGTCGGTTACGTGCGTTTATAT |
| | primer | (SEQ ID No. 7) |
| | Probe | CCCGTCGAAAACCCGCCGATTA |
| | | (SEQ ID No. 8) |
| COX2 | Sense | CGGAAGCGTTCGGGTAAAG |
| | primer | (SEQ ID No. 9) |
| | Antisense | AATTCCACCGCCCCAAAC |
| | primer | (SEQ ID No. 10) |
| | Probe | TTTCCGCCAAATATCTTTTCTTCTTCG |
| | | CA |
| | | (SEQ ID No. 11) |
| RASSF1A | Sense | GCGTTGAAGTCGGGGTTC |
| | primer | (SEQ ID No. 12) |
| | Antisense | AAACCCGTACTTCGCTAACTTTAAAC |
| | primer | (SEQ ID No. 13) |
| | Probe | ACAAACGCGAACCGAACGAAACCA |
| | | (SEQ ID No. 14) |
| β-actin | Sense | GGTGGAGGTAGTTAGGGTTTATTTGTA |
| | primer | (SEQ ID No. 15) |
| | Antisense | CCACACCACAAAATCACACTTAACCT |
| | primer | CATTT |
| | | (SEQ ID No. 16) |
| | Probe | CACTTTTATTCAACTAATCTC |
| | | (SEQ ID No. 17) |

B. Basic Statistics and ANOVA

Basic descriptive statistics of four genes APC, COX2, RASSF1A and miR-203 and individual differences among nine groups are presented as follows. Nine groups comprise group of healthy adult, group of infected with hepatitis virus B (HBV), group of infected with hepatitis virus C (HCV), group of infected with hepatitis virus B and cirrhosis (HBV+Cirrhosis), group of infected with hepatitis virus C and cirrhosis (HCV+Cirrhosis), group of liver cancer and hepatitis virus B (HCC−HBV), group of liver cancer and hepatitis virus C (HCC−HCV), group of liver cancer, hepatitis virus B and cirrhosis (HCC−HBV+Cirrhosis), and group of liver cancer, hepatitis virus C and cirrhosis (HCC−HCV+Cirrhosis).

(1) Basic Descriptive Statistics of APC Gene Methylation

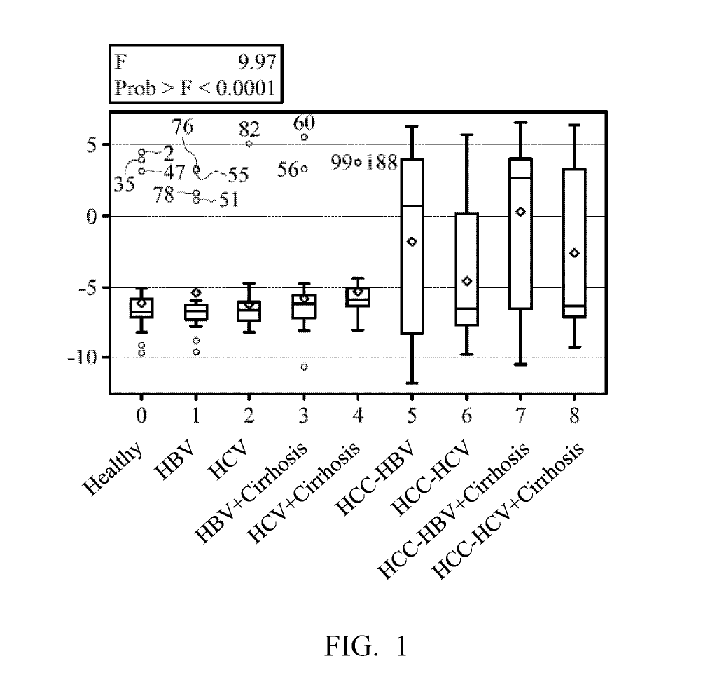

The basic descriptive statistics result of APC gene methylation is illustrated in Table 2 and FIG. 1.

TABLE 2
Variable Analysis: ln(APC)

| | No. | | | | | | | | | | Coefficient |
|---|---|---|---|---|---|---|---|---|---|---|---|
| | of | | | Std. | | | | Lower | Upper | Quartile | of |
| Groups | Objects | Mean | Median | Dev. | Range | Min | Max | Quartile | Quartile | Range | Variation |
| Healthy | 50 | −6.18 | −6.78 | 2.76 | 14.23 | −9.73 | 4.50 | −7.19 | −5.89 | 1.30 | −44.73 |
| HBV | 21 | −5.35 | −6.77 | 3.93 | 12.93 | −9.63 | 3.30 | −7.32 | −6.31 | 1.00 | −73.49 |
| HCV | 26 | −6.25 | −6.70 | 2.46 | 13.31 | −8.25 | 5.06 | −7.38 | −6.09 | 1.29 | −39.41 |
| HBV + | 32 | −5.85 | −6.21 | 2.95 | 16.18 | −10.63 | 5.55 | −7.22 | −5.61 | 1.61 | −50.43 |
| Cirrhosis | | | | | | | | | | | |
| HCV + | 25 | −5.26 | −5.96 | 2.88 | 11.88 | −8.05 | 3.83 | −6.36 | −5.09 | 1.27 | −54.84 |
| Cirrhosis | | | | | | | | | | | |
| HCC- | 81 | −1.79 | 0.71 | 6.29 | 18.14 | −11.84 | 6.30 | −8.26 | 4.00 | 12.26 | −351.80 |
| HBV | | | | | | | | | | | |
| HCC- | 30 | −4.63 | −6.55 | 4.70 | 15.55 | −9.81 | 5.74 | −7.70 | 0.16 | 7.86 | −101.47 |
| HCV | | | | | | | | | | | |
| HCC- | 42 | 0.26 | 2.62 | 5.33 | 17.11 | −10.49 | 6.62 | −6.57 | 4.08 | 10.65 | 2026.91 |
| HBV + | | | | | | | | | | | |
| Cirrhosis | | | | | | | | | | | |
| HCC- | 50 | −2.62 | −6.38 | 5.53 | 15.69 | −9.29 | 6.40 | −7.12 | 3.29 | 10.41 | −211.25 |
| HCV + | | | | | | | | | | | |
| Cirrhosis | | | | | | | | | | | |

After ANOVA analysis, the ln(APC) means of nine groups illustrate significantly different in statistics. Compared with non-HCC groups (including group of healthy adult, group of infected with HBV, group of infected with HCV, group of infected with HBV and Cirrhosis, group of infected with HCV and Cirrhosis), the methylation level of APC gene in HCC groups (including group of HCC−HBV, group of HCC−HCV, group of HCC−HBV and Cirrhosis, group of HCC−HCV and Cirrhosis) is significantly higher than that in non-HCC groups.

(2) Basic Descriptive Statistics of COX2 Gene Methylation

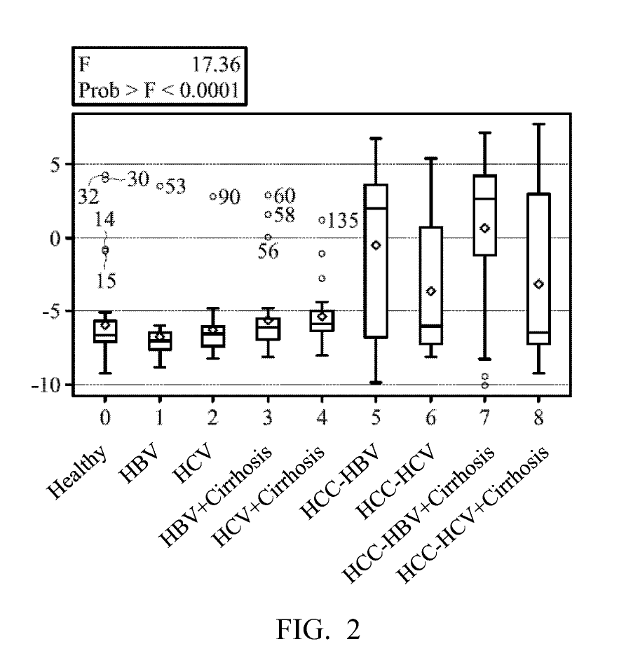

The basic descriptive statistics result of COX2 gene methylation is illustrated in Table 3 and FIG. 2.

TABLE 3
Variable Analysis: ln (COX2)

| | No. | | | | | | | | | | Coefficient |
|---|---|---|---|---|---|---|---|---|---|---|---|
| | of | | | Std. | | | | Lower | Upper | Quartile | of |
| Groups | Objects | Mean | Median | Dev. | Range | Min | Max | Quartile | Quartile | Range | Variation |
| Healthy | 50 | −5.96 | −6.68 | 2.51 | 13.49 | −9.25 | 4.24 | −7.13 | −5.67 | 1.46 | −42.06 |
| HBV | 21 | −6.69 | −7.03 | 2.48 | 12.34 | −8.82 | 3.52 | −7.64 | −6.45 | 1.20 | −37.03 |
| HCV | 26 | −6.32 | −6.58 | 2.05 | 11.05 | −8.25 | 2.81 | −7.38 | −6.09 | 1.29 | −32.44 |
| HBV + | 32 | −5.65 | −6.15 | 2.53 | 11.06 | −8.14 | 2.92 | −6.97 | −5.56 | 1.41 | −44.72 |
| Cirrhosis | | | | | | | | | | | |
| HCV + | 25 | −5.38 | −5.89 | 1.99 | 9.27 | −8.05 | 1.22 | −6.35 | −4.96 | 1.39 | −37.01 |
| Cirrhosis | | | | | | | | | | | |
| HCC- | 81 | −0.50 | 2.03 | 5.18 | 16.68 | −9.86 | 6.81 | −6.76 | 3.62 | 10.38 | −1034.86 |
| HBV | | | | | | | | | | | |
| HCC- | 30 | −3.61 | −6.07 | 4.58 | 13.55 | −8.13 | 5.42 | −7.22 | 0.69 | 7.91 | −126.95 |
| HCV | | | | | | | | | | | |
| HCC- | 42 | 0.66 | 2.64 | 5.03 | 17.23 | −10.10 | 7.14 | −1.17 | .30 | 5.47 | 756.28 |
| HBV + | | | | | | | | | | | |
| Cirrhosis | | | | | | | | | | | |
| HCC- | 50 | −3.11 | −6.46 | 5.42 | 16.96 | −9.25 | 7.71 | −7.23 | .93 | 10.16 | −174.17 |
| HCV + | | | | | | | | | | | |
| Cirrhosis | | | | | | | | | | | |

After ANOVA analysis, the ln(COX2) means of nine groups illustrate significantly different in statistics. Compared with non-HCC groups (including group of healthy adult, group of infected with HBV, group of infected with HCV, group of infected with HBV and Cirrhosis, group of infected with HCV and Cirrhosis), the methylation level of COX2 gene in HCC groups (including group of HCC−HBV, group of HCC−HCV, group of HCC−HBV and Cirrhosis, group of HCC−HCV and Cirrhosis) is significantly higher than that in non-HCC groups.

(3) Basic Descriptive Statistics of miR-203 Gene Methylation

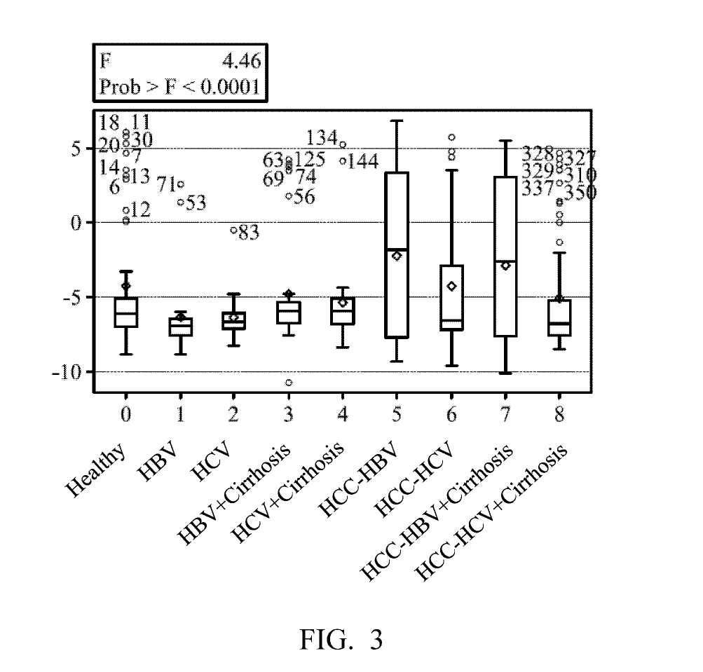

The basic descriptive statistics result of miR-203 gene methylation is illustrated in Table 4 and FIG. 3.

TABLE 4
Variable Analysis: ln (miR-203)

| | No. | | | | | | | | | | Coefficient |
|---|---|---|---|---|---|---|---|---|---|---|---|
| | of | | | Std. | | | | Lower | Upper | Quartile | of |
| Groups | Objects | Mean | Median | Dev. | Range | Min | Max | Quartile | Quartile | Range | Variation |
| Healthy | 50 | −4.23 | −6.11 | 4.31 | 14.86 | −8.85 | 6.01 | −6.93 | −5.08 | 1.84 | −101.89 |
| HBV | 21 | −6.32 | −6.89 | 2.88 | 11.42 | −8.82 | 2.60 | −7.53 | −6.45 | 1.09 | −45.64 |
| HCV | 26 | −6.41 | −6.67 | 1.46 | 7.76 | −8.25 | −0.49 | −7.09 | −6.09 | 1.00 | −22.79 |
| HBV + | 32 | −4.82 | −5.92 | 3.77 | 14.89 | −10.74 | 4.15 | −6.74 | −5.35 | 1.38 | −78.28 |
| Cirrhosis | | | | | | | | | | | |
| HCV + | 25 | −5.31 | −5.96 | 3.19 | 13.52 | −8.30 | 5.22 | −6.75 | −5.09 | 1.66 | −60.03 |
| Cirrhosis | | | | | | | | | | | |
| HCC- | 81 | −2.23 | −1.78 | 5.40 | 16.15 | −9.31 | 6.84 | −7.64 | 3.34 | 10.99 | −242.39 |
| HBV | | | | | | | | | | | |
| HCC- | 30 | −4.28 | −6.55 | 4.76 | 15.29 | −9.59 | 5.70 | −7.14 | −2.84 | 4.30 | −111.31 |
| HCV | | | | | | | | | | | |
| HCC- | 42 | −2.87 | −2.59 | 5.15 | 15.55 | −10.10 | 5.45 | −7.61 | 3.06 | 10.68 | −179.70 |
| HBV + | | | | | | | | | | | |
| Cirrhosis | | | | | | | | | | | |
| HCC- | 50 | −5.03 | −6.73 | 4.05 | 12.98 | −8.53 | 4.45 | −7.55 | −5.24 | 2.31 | −80.58 |
| HCV + | | | | | | | | | | | |
| Cirrhosis | | | | | | | | | | | |

After ANOVA analysis, the ln(miR-203) means of nine groups illustrate significantly different in statistics. Compared with non-HCC groups (including group of healthy adult, group of infected with HBV, group of infected with HCV, group of infected with HBV and Cirrhosis, group of infected with HCV and Cirrhosis), the methylation level of miR-203 gene in HCC groups (including group of HCC−HBV, group of HCC−HCV, group of HCC−HBV and Cirrhosis, group of HCC−HCV and Cirrhosis) is significantly higher than that in non-HCC groups.

(4) Basic Descriptive Statistics of RASSF1A Gene Methylation

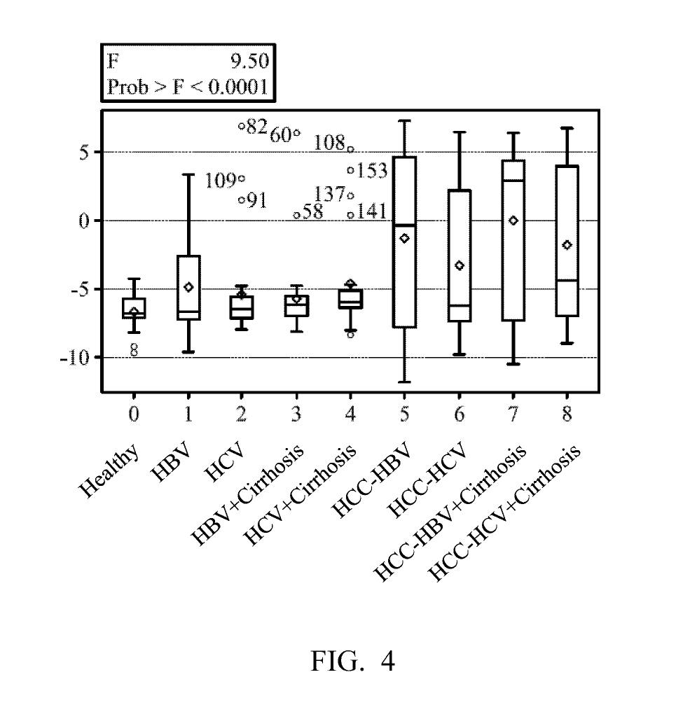

The basic descriptive statistics result of RASSF1A gene methylation is illustrated in Table 5 and FIG. 4.

TABLE 5
Variable Analysis: ln (RASSF1A)

| | No. | | | | | | | | | | Coefficient |
|---|---|---|---|---|---|---|---|---|---|---|---|
| | of | | | Std. | | | | Lower | Upper | Quartile | of |
| Groups | Objects | Mean | Median | Dev. | Range | Min | Max | Quartile | Quartile | Range | Variation |
| Healthy | 50 | −6.63 | −6.74 | 1.04 | 5.40 | −9.68 | −4.28 | −7.14 | −5.95 | 1.19 | −15.69 |
| HBV | 21 | −4.91 | −6.67 | 3.99 | 13.02 | −9.63 | 3.38 | −7.19 | −2.59 | 4.60 | −81.32 |
| HCV | 26 | −5.42 | −6.48 | 3.60 | 14.89 | −7.95 | 6.94 | −7.14 | −5.59 | 1.55 | −66.30 |
| HBV + | 32 | −5.73 | −6.15 | 2.68 | 14.58 | −8.14 | 6.44 | −6.97 | −5.56 | 1.41 | −46.84 |
| Cirrhosis | | | | | | | | | | | |
| HCV + | 25 | −4.60 | −5.99 | 3.75 | 13.52 | −8.30 | 5.22 | −6.36 | −5.09 | 1.27 | −81.51 |
| Cirrhosis | | | | | | | | | | | |
| HCC- | 81 | −1.30 | −0.32 | 6.22 | 19.17 | −11.84 | 7.33 | −7.82 | 4.69 | 12.51 | −479.37 |
| HBV | | | | | | | | | | | |
| HCC- | 30 | −3.31 | −6.19 | 5.31 | 16.24 | −9.81 | 6.43 | −7.37 | 2.20 | 9.57 | −160.19 |
| HCV | | | | | | | | | | | |
| HCC- | 42 | −0.06 | 2.92 | 5.82 | 16.96 | −10.49 | 6.47 | −7.34 | 4.39 | 11.73 | −9335.44 |
| HBV + | | | | | | | | | | | |
| Cirrhosis | | | | | | | | | | | |
| HCC- | 50 | −1.76 | −4.39 | 5.72 | 15.78 | −8.95 | 6.83 | −6.96 | 4.05 | 11.01 | −324.41 |
| HCV + | | | | | | | | | | | |
| Cirrhosis | | | | | | | | | | | |

After ANOVA analysis, the ln(RASSF1A) means of nine groups illustrate significantly different in statistics. Compared with non-HCC groups (including group of healthy adult, group of infected with HBV, group of infected with HCV, group of infected with HBV and Cirrhosis, group of infected with HCV and Cirrhosis), the methylation level of RASSF1A gene in HCC groups (including group of HCC−HBV, group of HCC−HCV, group of HCC−HBV and Cirrhosis, group of HCC−HCV and Cirrhosis) is significantly higher than that in non-HCC groups.

C. Receiver Operating Characteristic Curve (ROC Curve)

Prediction of the Risk for Suffering from Liver Cancer

The connection between gene and liver cancer is utilized to perform model prediction of logistic regression for finding the prediction probability of liver cancer as the best cut point with better sensitivity and accuracy. After performing with receiver operating characteristic curve (ROC curve) and estimation of the area, the distinction ability of prediction model to hepatocellular carcinoma is evaluated.

Nine Groups as Follows:

Non-HCC groups comprise: group of healthy adult, group of infected with HBV, group of infected with HCV, group of infected with HBV and Cirrhosis, group of infected with HCV and Cirrhosis (The number of subjects is 154, N=154), and HCC groups comprise: group of HCC−HBV, group of HCC−HCV, group of HCC−HBV and cirrhosis, and group of HCC−HCV and Cirrhosis (The number of subjects is 203, N=203).

1. Single Methylation Markers for Prediction of Liver Cancer

(1) APC

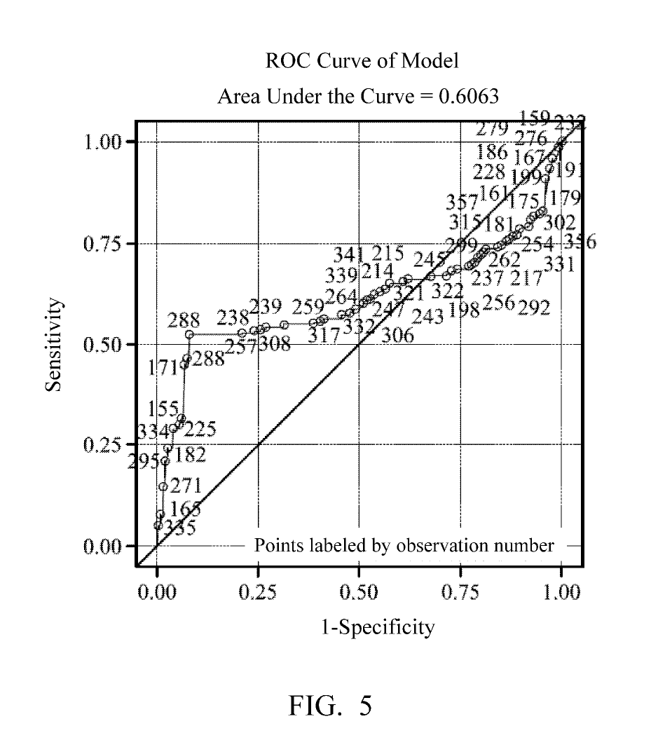

The prediction model of ln(APC) of above-mentioned nine groups is established as Ln(P/(1−P))=0.9753+0.1683×ln(APC). ROC curve analysis is performed next and the result is illustrated in FIG. 5. According to the prediction model of APC gene in FIG. 5, the area of ROC Curve is 0.6063. Moreover, while the best cut-off value is 0.48, the sensitivity is 56.2%, the specificity is 57.1%, and the overall accuracy is 56.6%.

(2) COX2

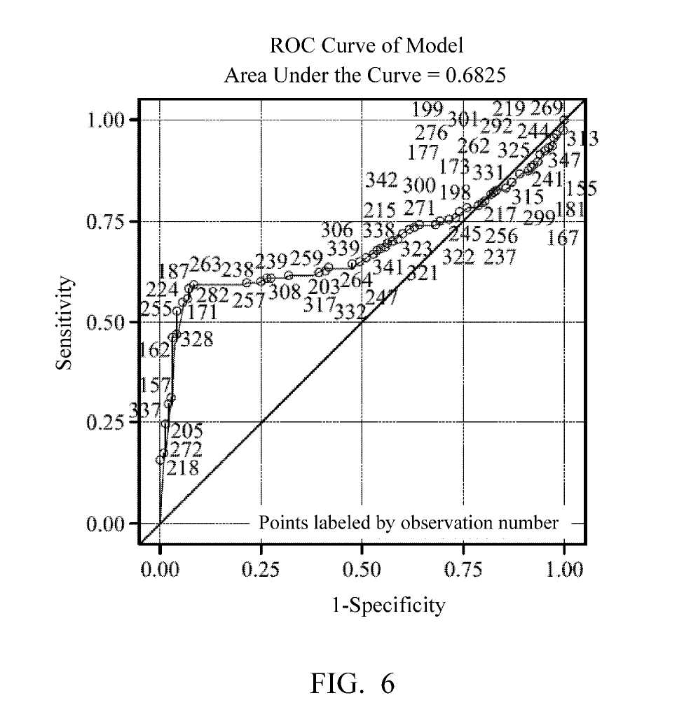

The prediction model of ln(COX2) of above-mentioned nine groups is established as Ln(P/(1−P))=1.2778+0.2479×ln(COX2). ROC curve analysis is performed next and the result is illustrated in FIG. 6. According to the prediction model of COX2 gene in FIG. 6, the area of ROC Curve is 0.683. Moreover, while the best cut-off value is 0.45, the sensitivity is 61.1%, the specificity is 66.2%, and the overall accuracy is 63.3%.

(3) miR-203

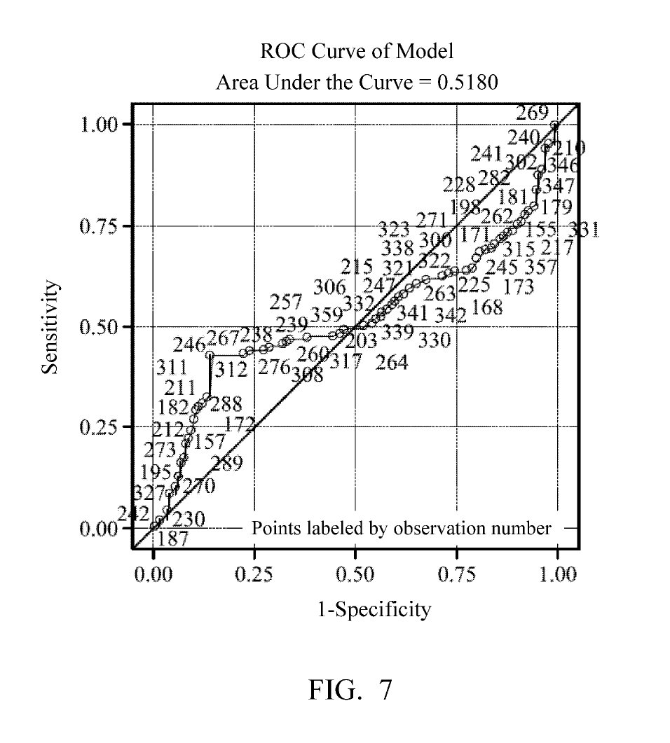

The prediction model of ln(miR-203) of above-mentioned nine groups is established as Ln(P/(1−P))=0.6845+0.0942×ln(miR-203). ROC curve analysis is performed next and the result is illustrated in FIG. 7. According to the prediction model of miR-203 gene in FIG. 7, the area of ROC Curve is 0.518. Moreover, while the best cut-off value is 0.52, the sensitivity is 49.3%, the specificity is 43.5%, and the overall accuracy is 46.8%.

(4) RASSF1A

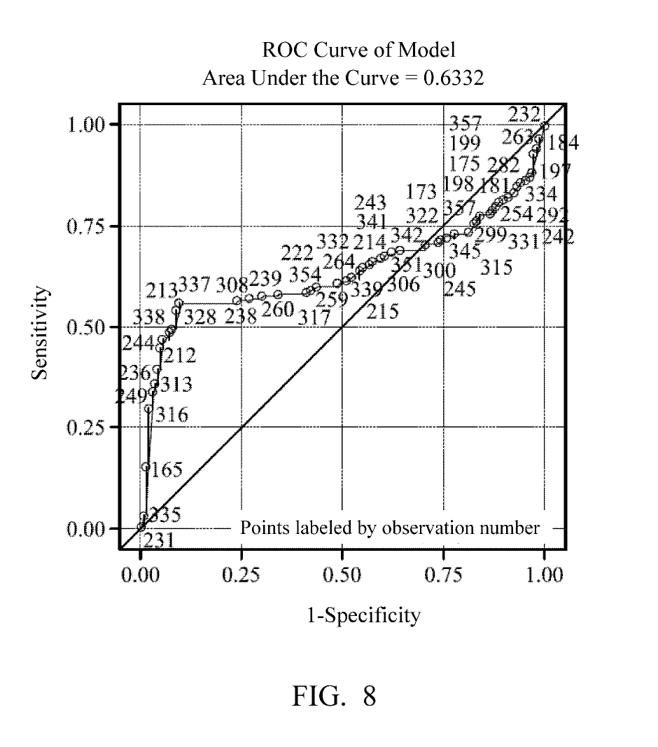

The prediction model of ln(RASSF1A) of above-mentioned nine groups is established as Ln(P/(1−P))=0.9818+0.1787×ln(RASSF1A). ROC curve analysis is performed next and the result is illustrated in FIG. 8. According to the prediction model of RASSF1A gene in FIG. 8, the area of ROC Curve is 0.6332. Moreover, while the best cut-off value is 0.46, the sensitivity is 59.8%, the specificity is 48.6%, and the overall accuracy is 55.0%.

2. Multiple Methylation Markers for Prediction of Liver Cancer

(1) Stepwise Selection

Ln(APC), ln(COX2), In(RASSF1A) and ln(miR-203) of the above-mentioned nine groups are performed by Stepwise selection analysis, in which these four factors enter the model in the order of ln(COX2), In(RASSF1A), In(APC) and In(miR-203), and no factor is removed.

(2) Maximum Likelihood Estimates

Ln(APC), ln(COX2), ln(RASSF1A) and In(miR-203) of the above-mentioned nine groups are performed by Maximum Likelihood Estimates, Parameter Estimation, and analysis of Wald confidence interval, and the results are illustrated as TABLE 6.

TABLE 6

| | | | Standard | Wald | Pr > Chi- | 95% Confidence | |
|---|---|---|---|---|---|---|---|
| Parameter | DF | Estimate | Error | Chi-Square | Square | Limits | |
| Intercept | 1 | 2.2383 | 0.3181 | 49.5102 | <.0001 | 1.6148 | 2.8618 |
| ln (APC) | 1 | 0.0898 | 0.0337 | 7.1037 | 0.0077 | 0.0237 | 0.1559 |
| ln (COX2) | 1 | 0.1875 | 0.0360 | 27.1781 | <.0001 | 0.1169 | 0.2581 |
| ln (miR-203) | 1 | 0.0701 | 0.0328 | 4.5693 | 0.0325 | 0.0058 | 0.1344 |
| ln (RASSFIA34) | 1 | 0.1097 | 0.0337 | 10.5752 | 0.0011 | 0.0436 | 0.1758 |

(3) Odds Ratio Estimates and Profile-Likelihood Confidence Intervals

Ln(APC), ln(COX2), ln(RASSF1A) and ln(miR-203) of the above-mentioned nine groups are performed by Odds Ratio Estimates and Profile-Likelihood Confidence Intervals, and the results are illustrated as TABLE 7.

TABLE 7

| Effect | Unit | Estimate | 95% Confidence Limits | |
|---|---|---|---|---|
| ln (APC) | 1.0000 | 1.094 | 1.025 | 1.170 |
| ln (COX2) | 1.0000 | 1.206 | 1.127 | 1.299 |
| ln (miRNA-203) | 1.0000 | 1.073 | 1.006 | 1.145 |
| ln (RASSF1A) | 1.0000 | 1.116 | 1.045 | 1.194 |

According to Table 7, the odds ratio in the risk of suffering from HCC increases 9.4% when ln(APC) rises in per one unit, the odds ratio in the risk of suffering from HCC increases 20.6% when ln(COX2) rises in per one unit, the odds ratio in the risk of suffering from HCC increases 7.3% when ln(miRNA-203) rises in per one unit, and the odds ratio in the risk of suffering from HCC increases 11.6% when ln(RASSF1A) rises in per one unit, in which the degree of methylation in COX2 is the most influential among the four above-mentioned genes.

After foregoing analysis, stepwise regression analysis is performed to select the four variables ln(APC), ln(COX2), ln(miR-203) and ln(RASSF1A) to establish the model as follows.

Prediction model A: Ln(P/(1−P))=2.238+0.0898×ln(APC)+0.1875×In(COX2)+0.0701×ln(miRNA-203)+0.1097×ln(RASSF1A)

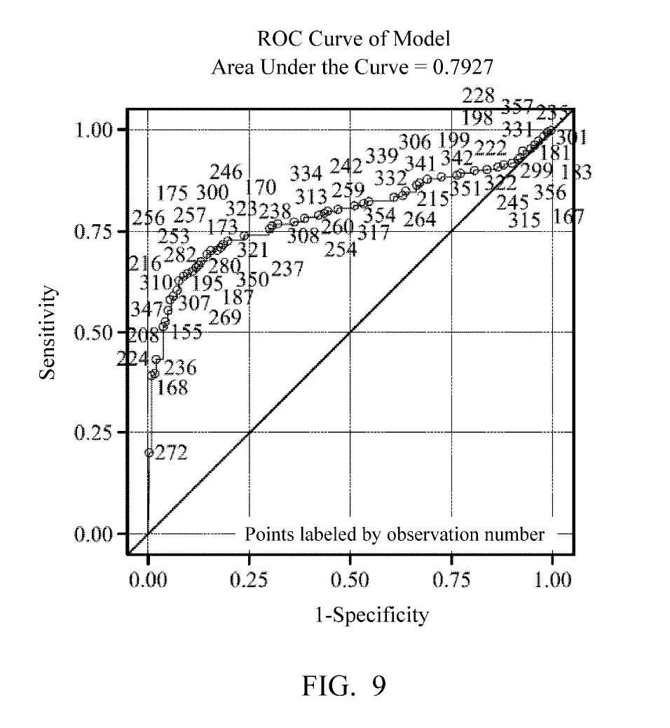

Then, ROC curve analysis is performed and the results are illustrated as TABLE 8 and FIG. 9.

TABLE 8
The relationship among cutoff value of ROC curve, sensitivity,
specificity, false positives, false negatives and overall accuracy

| | Correct | | Incorrect | | percentage | | | | |
|---|---|---|---|---|---|---|---|---|---|
| Cut-off | | Non- | | Non- | | | | False | False |
| Value | Event | event | Event | event | Correct | Sensitivity | Specificity | POS | NEG |
| 0.350 | 164 | 66 | 82 | 35 | 66.3 | 82.4 | 44.6 | 33.3 | 34.7 |
| 0.360 | 160 | 69 | 79 | 39 | 66.0 | 80.4 | 46.6 | 33.1 | 36.1 |
| 0.370 | 157 | 76 | 72 | 42 | 67.1 | 78.9 | 51.4 | 31.4 | 35.6 |
| 0.380 | 155 | 83 | 65 | 44 | 68.6 | 77.9 | 56.1 | 29.5 | 34.6 |
| 0.390 | 155 | 84 | 64 | 44 | 68.9 | 77.9 | 56.8 | 29.2 | 34.4 |
| 0.400 | 154 | 91 | 57 | 45 | 70.6 | 77.4 | 61.5 | 27.0 | 33.1 |
| 0.410 | 154 | 92 | 56 | 45 | 70.9 | 77.4 | 62.2 | 26.7 | 32.8 |
| 0.420 | 152 | 95 | 53 | 47 | 71.2 | 76.4 | 64.2 | 25.9 | 33.1 |
| 0.430 | 147 | 101 | 47 | 52 | 71.5 | 73.9 | 68.2 | 24.2 | 34.0 |
| 0.440 | 147 | 104 | 44 | 52 | 72.3 | 73.9 | 70.3 | 23.0 | 33.3 |
| 0.450 | 146 | 108 | 40 | 53 | 73.2 | 73.4 | 73.0 | 21.5 | 32.9 |
| 0.460 | 145 | 111 | 37 | 54 | 73.8 | 72.9 | 75.0 | 20.3 | 32.7 |
| 0.470 | 145 | 114 | 34 | 54 | 74.6 | 72.9 | 77.0 | 19.0 | 32.1 |
| 0.480 | 143 | 115 | 33 | 56 | 74.4 | 71.9 | 77.7 | 18.8 | 32.7 |
| 0.490 | 142 | 117 | 31 | 57 | 74.6 | 71.4 | 79.1 | 17.9 | 32.8 |
| 0.500 | 140 | 119 | 29 | 59 | 74.6 | 70.4 | 80.4 | 17.2 | 33.1 |
| 0.510 | 139 | 120 | 28 | 60 | 74.6 | 69.8 | 81.1 | 16.8 | 33.3 |
| 0.520 | 138 | 123 | 25 | 61 | 75.2 | 69.3 | 83.1 | 15.3 | 33.2 |
| 0.530 | 137 | 124 | 24 | 62 | 75.2 | 68.8 | 83.8 | 14.9 | 33.3 |
| 0.540 | 134 | 126 | 22 | 65 | 74.9 | 67.3 | 85.1 | 14.1 | 34.0 |
| 0.550 | 133 | 126 | 22 | 66 | 74.6 | 66.8 | 85.1 | 14.2 | 34.4 |
| 0.560 | 132 | 127 | 21 | 67 | 74.6 | 66.3 | 85.8 | 13.7 | 34.5 |
| 0.570 | 131 | 128 | 20 | 68 | 74.6 | 65.8 | 86.5 | 13.2 | 34.7 |
| 0.580 | 129 | 129 | 19 | 70 | 74.4 | 64.8 | 87.2 | 12.8 | 35.2 |
| 0.590 | 128 | 129 | 19 | 71 | 74.1 | 64.3 | 87.2 | 12.9 | 35.5 |
| 0.600 | 128 | 130 | 18 | 71 | 74.4 | 64.3 | 87.8 | 12.3 | 35.3 |
| 0.610 | 127 | 131 | 17 | 72 | 74.4 | 63.8 | 88.5 | 11.8 | 35.5 |
| 0.620 | 127 | 133 | 15 | 72 | 74.9 | 63.8 | 89.9 | 10.6 | 35.1 |
| 0.630 | 126 | 134 | 14 | 73 | 74.9 | 63.3 | 90.5 | 10.0 | 35.3 |
| 0.640 | 126 | 135 | 13 | 73 | 75.2 | 63.3 | 91.2 | 9.4 | 35.1 |
| 0.650 | 125 | 135 | 13 | 74 | 74.9 | 62.8 | 91.2 | 9.4 | 35.4 |
| 0.660 | 125 | 135 | 13 | 74 | 74.9 | 62.8 | 91.2 | 9.4 | 35.4 |
| 0.670 | 125 | 136 | 12 | 74 | 75.2 | 62.8 | 91.9 | 8.8 | 35.2 |
| 0.680 | 125 | 137 | 11 | 74 | 75.5 | 62.8 | 92.6 | 8.1 | 35.1 |
| 0.690 | 122 | 137 | 11 | 77 | 74.6 | 61.3 | 92.6 | 8.3 | 36.0 |
| 0.700 | 118 | 137 | 11 | 81 | 73.5 | 59.3 | 92.6 | 8.5 | 37.2 |
| 0.710 | 114 | 137 | 11 | 85 | 72.3 | 57.3 | 92.6 | 8.8 | 38.3 |
| 0.720 | 113 | 137 | 11 | 86 | 72.0 | 56.8 | 92.6 | 8.9 | 38.6 |
| 0.730 | 111 | 138 | 10 | 88 | 71.8 | 55.8 | 93.2 | 8.3 | 38.9 |
| 0.740 | 110 | 140 | 8 | 89 | 72.0 | 55.3 | 94.6 | 6.8 | 38.9 |
| 0.750 | 105 | 140 | 8 | 94 | 70.6 | 52.8 | 94.6 | 7.1 | 40.2 |
| 0.760 | 102 | 140 | 8 | 97 | 69.7 | 51.3 | 94.6 | 7.3 | 40.9 |
| 0.770 | 102 | 141 | 7 | 97 | 70.0 | 51.3 | 95.3 | 6.4 | 40.8 |
| 0.780 | 102 | 141 | 7 | 97 | 70.0 | 51.3 | 95.3 | 6.4 | 40.8 |
| 0.790 | 98 | 143 | 5 | 101 | 69.5 | 49.2 | 96.6 | 4.9 | 41.4 |
| 0.800 | 98 | 143 | 5 | 101 | 69.5 | 49.2 | 96.6 | 4.9 | 41.4 |

According to TABLE 8 and FIG. 9, area of ROC curve is 0.793, indicating that a better classification result is obtained by performing prediction model A to classify Non-HCC groups and HCC groups. When the best cutoff value of model is 0.45, the sensitivity of 73.4%, specificity of 73.0%, false positives of 21.5%, false negatives of 32.9% as well as overall accuracy of 73.2% are obtained.

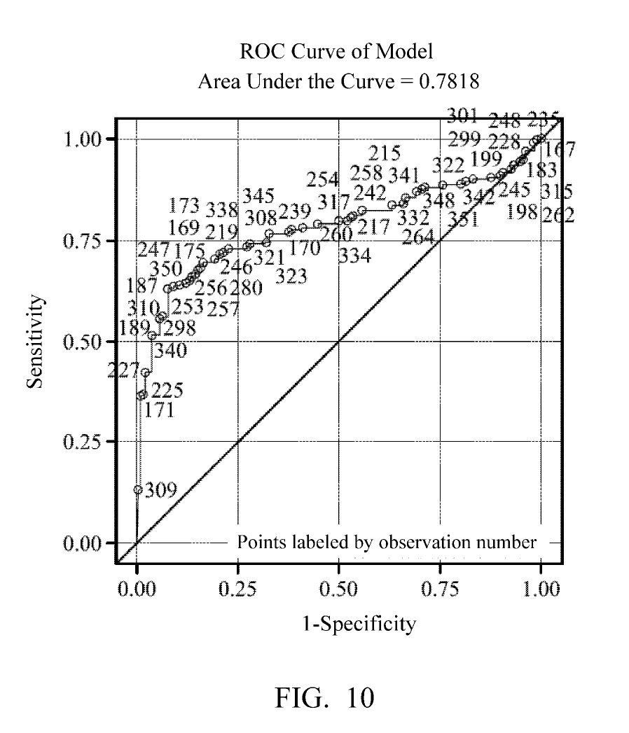

In addition, the method of Leave-one-out cross-validation (LOOCV) is performed to verify the model and used for confirming the classification capacity of this model. The results is illustrated in Table 9 and FIG. 10.

TABLE 9
The relationship among cutoff value, sensitivity, specificity, false
positives, false negatives and overall accuracy of ROC curve

| | Correct | | Incorrect | | percentage | | | | |
|---|---|---|---|---|---|---|---|---|---|
| Cut-off | | Non- | | Non- | | | | False | False |
| Value | Event | event | Event | event | Correct | Sensitivity | Specificity | POS | NEG |
| 0.300 | 173 | 42 | 106 | 26 | 62.0 | 86.9 | 28.4 | 38.0 | 38.2 |
| 0.310 | 170 | 46 | 102 | 29 | 62.2 | 85.4 | 31.1 | 37.5 | 38.7 |
| 0.320 | 167 | 50 | 98 | 32 | 62.5 | 83.9 | 33.8 | 37.0 | 39.0 |
| 0.330 | 164 | 54 | 94 | 35 | 62.8 | 82.4 | 36.5 | 36.4 | 39.3 |
| 0.340 | 164 | 58 | 90 | 35 | 64.0 | 82.4 | 39.2 | 35.4 | 37.6 |
| 0.350 | 161 | 64 | 84 | 38 | 64.8 | 80.9 | 43.2 | 34.3 | 37.3 |
| 0.360 | 158 | 68 | 80 | 41 | 65.1 | 79.4 | 45.9 | 33.6 | 37.6 |
| 0.370 | 157 | 73 | 75 | 42 | 66.3 | 78.9 | 49.3 | 32.3 | 36.5 |
| 0.380 | 155 | 77 | 71 | 44 | 66.9 | 77.9 | 52.0 | 31.4 | 36.4 |
| 0.390 | 155 | 84 | 64 | 44 | 68.9 | 77.9 | 56.8 | 29.2 | 34.4 |
| 0.400 | 154 | 85 | 63 | 45 | 68.9 | 77.4 | 57.4 | 29.0 | 34.6 |
| 0.410 | 154 | 91 | 57 | 45 | 70.6 | 77.4 | 61.5 | 27.0 | 33.1 |
| 0.420 | 153 | 92 | 56 | 46 | 70.6 | 76.9 | 62.2 | 26.8 | 33.3 |
| 0.430 | 152 | 94 | 54 | 47 | 70.9 | 76.4 | 63.5 | 26.2 | 33.3 |
| 0.440 | 148 | 100 | 48 | 51 | 71.5 | 74.4 | 67.6 | 24.5 | 33.8 |
| 0.450 | 147 | 103 | 45 | 52 | 72.0 | 73.9 | 69.6 | 23.4 | 33.5 |
| 0.460 | 147 | 104 | 44 | 52 | 72.3 | 73.9 | 70.3 | 23.0 | 33.3 |
| 0.470 | 145 | 108 | 40 | 54 | 72.9 | 72.9 | 73.0 | 21.6 | 33.3 |
| 0.480 | 145 | 111 | 37 | 54 | 73.8 | 72.9 | 75.0 | 20.3 | 32.7 |
| 0.490 | 145 | 113 | 35 | 54 | 74.4 | 72.9 | 76.4 | 19.4 | 32.3 |
| 0.500 | 144 | 115 | 33 | 55 | 74.6 | 72.4 | 77.7 | 18.6 | 32.4 |
| 0.510 | 143 | 117 | 31 | 56 | 74.9 | 71.9 | 79.1 | 17.8 | 32.4 |
| 0.520 | 141 | 117 | 31 | 58 | 74.4 | 70.9 | 79.1 | 18.0 | 33.1 |
| 0.530 | 140 | 119 | 29 | 59 | 74.6 | 70.4 | 80.4 | 17.2 | 33.1 |
| 0.540 | 138 | 120 | 28 | 61 | 74.4 | 69.3 | 81.1 | 16.9 | 33.7 |
| 0.550 | 138 | 123 | 25 | 61 | 75.2 | 69.3 | 83.1 | 15.3 | 33.2 |
| 0.560 | 138 | 124 | 24 | 61 | 75.5 | 69.3 | 83.8 | 14.8 | 33.0 |
| 0.570 | 135 | 124 | 24 | 64 | 74.6 | 67.8 | 83.8 | 15.1 | 34.0 |
| 0.580 | 134 | 126 | 22 | 65 | 74.9 | 67.3 | 85.1 | 14.1 | 34.0 |
| 0.590 | 133 | 126 | 22 | 66 | 74.6 | 66.8 | 85.1 | 14.2 | 34.4 |
| 0.600 | 132 | 127 | 21 | 67 | 74.6 | 66.3 | 85.8 | 13.7 | 34.5 |
| 0.610 | 131 | 127 | 21 | 68 | 74.4 | 65.8 | 85.8 | 13.8 | 34.9 |
| 0.620 | 129 | 128 | 20 | 70 | 74.1 | 64.8 | 86.5 | 13.4 | 35.4 |
| 0.630 | 128 | 129 | 19 | 71 | 74.1 | 64.3 | 87.2 | 12.9 | 35.5 |
| 0.640 | 128 | 129 | 19 | 71 | 74.1 | 64.3 | 87.2 | 12.9 | 35.5 |
| 0.650 | 127 | 130 | 18 | 72 | 74.1 | 63.8 | 87.8 | 12.4 | 35.6 |
| 0.660 | 127 | 131 | 17 | 72 | 74.4 | 63.8 | 88.5 | 11.8 | 35.5 |
| 0.670 | 127 | 133 | 15 | 72 | 74.9 | 63.8 | 89.9 | 10.6 | 35.1 |
| 0.680 | 126 | 133 | 15 | 73 | 74.6 | 63.3 | 89.9 | 10.6 | 35.4 |
| 0.690 | 126 | 135 | 13 | 73 | 75.2 | 63.3 | 91.2 | 9.4 | 35.1 |
| 0.700 | 125 | 135 | 13 | 74 | 74.9 | 62.8 | 91.2 | 9.4 | 35.4 |
| 0.710 | 125 | 135 | 13 | 74 | 74.9 | 62.8 | 91.2 | 9.4 | 35.4 |
| 0.720 | 125 | 136 | 12 | 74 | 75.2 | 62.8 | 91.9 | 8.8 | 35.2 |
| 0.730 | 124 | 137 | 11 | 75 | 75.2 | 62.3 | 92.6 | 8.1 | 35.4 |
| 0.740 | 122 | 137 | 11 | 77 | 74.6 | 61.3 | 92.6 | 8.3 | 36.0 |
| 0.750 | 117 | 137 | 11 | 82 | 73.2 | 58.8 | 92.6 | 8.6 | 37.4 |
| 0.760 | 114 | 137 | 11 | 85 | 72.3 | 57.3 | 92.6 | 8.8 | 38.3 |
| 0.770 | 113 | 137 | 11 | 86 | 72.0 | 56.8 | 92.6 | 8.9 | 38.6 |
| 0.780 | 110 | 137 | 11 | 89 | 71.2 | 55.3 | 92.6 | 9.1 | 39.4 |
| 0.790 | 106 | 140 | 8 | 93 | 70.9 | 53.3 | 94.6 | 7.0 | 39.9 |
| 0.800 | 102 | 140 | 8 | 97 | 69.7 | 51.3 | 94.6 | 7.3 | 40.9 |

According to TABLE 9 and FIG. 10, when the method of Leave-one-out cross-validation (LOOCV) is performed to verify the model, the area of ROC curve is 0.7818. When the best cutoff value of model is 0.47, the sensitivity of 72.9%, specificity of 73%, false positives of 21.6%, false negatives of 33.3% as well as overall accuracy of 72.9% are obtained.

3. AFP Marker for Prediction of Liver Cancer

Ln(AFP) of the above-mentioned nine groups is performed to establish the model as follows.

Prediction model: ln(P/(1−P))=0.7865+0.1198×ln(AFP)

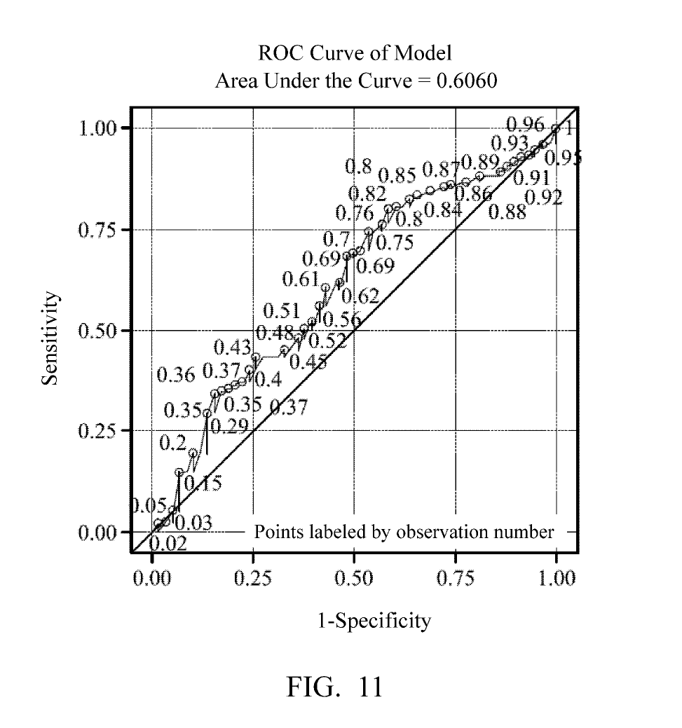

Then, ROC curve analysis is performed and the results are illustrated as TABLE 10 and FIG. 11. When the best cutoff value of model is 0.75, the sensitivity of 55.7%, specificity of 56.9%, false positives of 18.8%, false negatives of 72.3% as well as overall accuracy of 56.0% are obtained.

TABLE 10
When AFP is performed by ROC curve analysis and the best cut-
off value is 0.75, the sensitivity, specificity, false positives,
false negatives and overall accuracy are as follows.

| | AFP (ng/ml) | | | Sensitivity | 55.7 |
|---|---|---|---|---|---|
| | >13.5 | ≦13.5 | Total | Specificity | 56.9 |
| HCC Cases | 108 | 33 | 141 | False POS | 18.8 |
| Non-HCC Cases | 25 | 86 | 111 | False NEG | 72.3 |
| Total | 133 | 119 | 252 | Correct | 56.0 |

Prediction of the Risk Suffering from Hepatitis B-Related Liver Cancer

Five groups as follows are evaluated: non-HCC groups comprise group of healthy adult, group of infected with HBV, and group of infected with HBV and Cirrhosis (The number of subjects is 100, N=100), and HCC groups comprise group of HCC−HBV, and group of HCC−HBV and Cirrhosis (The number of subjects is 120, N=120).

1. Single Methylation Markers for Prediction of Hepatitis B-Related Liver Cancer

(1) APC

Ln(APC) of the above-mentioned five groups is performed to establish the model as follows.

Prediction model: ln(P/(1−P))=0.9165+0.1922×ln(APC)

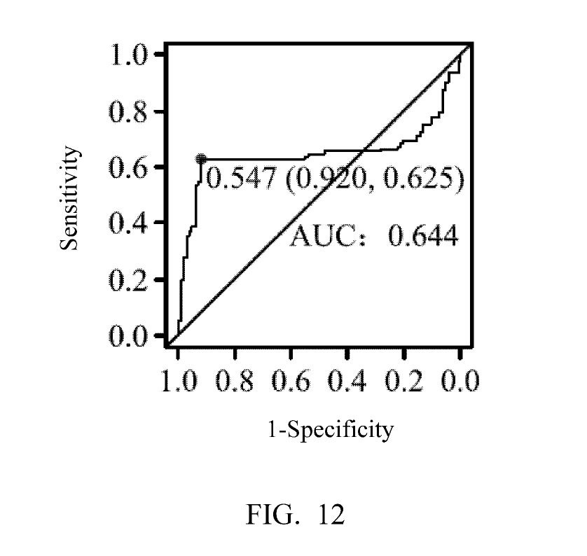

Then, ROC curve analysis is performed and the results are illustrated as FIG. 12. According to FIG. 12, the area of ROC curve is 0.644 in the prediction model of APC. When the best cutoff value of model is 0.547, the sensitivity of 62.5%, specificity of 92% as well as overall accuracy of 75.9% are obtained.

(2) COX2

Ln(COX2) of the above-mentioned five groups is performed to establish the model as follows.

Prediction model: ln(P/(1−P))=1.20072+0.29966×ln(COX2)

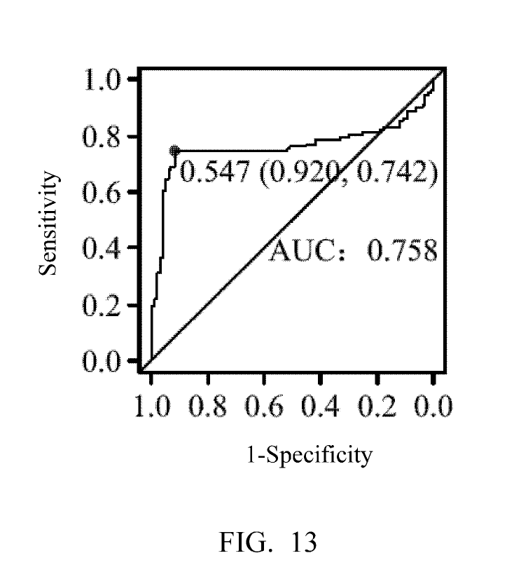

Then, ROC curve analysis is performed and the results are illustrated as FIG. 13. According to FIG. 13, the area of ROC curve is 0.758 in the prediction model of COX2. When the best cutoff value of model is 0.454, the sensitivity of 74.16%, specificity of 92% as well as overall accuracy of 82.27% are obtained.

(3) miR-203

Ln(miR-203) of the above-mentioned five groups is performed to establish the model as follows.

Prediction model: ln(P/(1−P))=0.5909+0.1096×ln(miR-203)

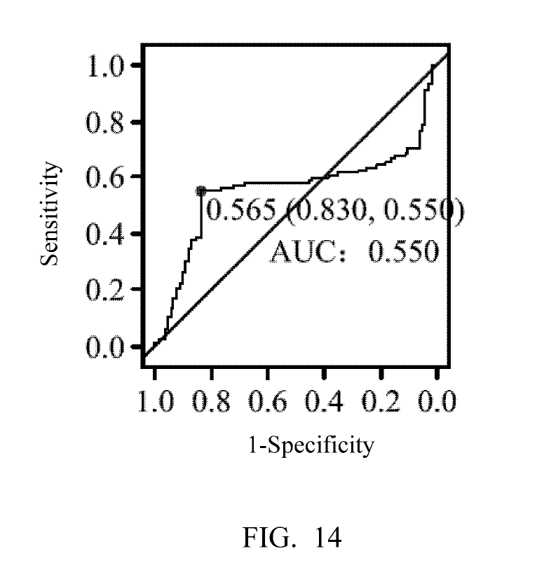

Then, ROC curve analysis is performed and the results are illustrated as FIG. 14. According to FIG. 14, the area of ROC curve is 0.55 in the prediction model of APC. When the best cutoff value of model is 0.565, the sensitivity of 55%, specificity of 83% as well as overall accuracy of 67.73% are obtained.

(4) RASSF1A

Ln(RASSF1A) of the above-mentioned five groups is performed to establish the model as follows.

Prediction model: ln(P/(1−P))=0.99403+0.21392×ln(RASSF1A)

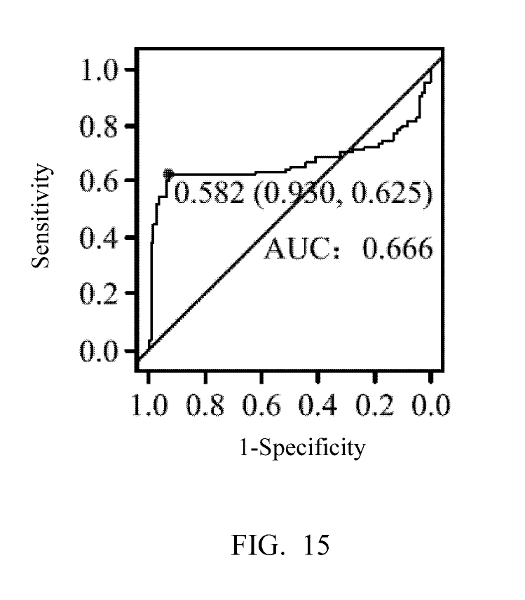

Then, ROC curve analysis is performed and the results are illustrated as FIG. 15. According to FIG. 15, the area of ROC curve is 0.67 in the prediction model of APC. When the best cutoff value of model is 0.582, the sensitivity of 62.5%, specificity of 83% as well as overall accuracy of 76.36% are obtained.

2. Multiple Methylation Markers for Prediction of Hepatitis B-Related Liver Cancer

(1) Stepwise Selection

Ln(APC), ln(COX2), ln(RASSF1A) and ln(miR-203) of the above-mentioned five groups are performed by Stepwise selection analysis, in which these four factors enter the model in the order of ln(COX2), ln(RASSF1A), ln(APC) and ln(miR-203), and no factor is removed.

(2) Maximum Likelihood Estimates

Ln(APC), ln(COX2), ln(RASSF1A) and ln(miR-203) of the above-mentioned five groups are performed by Maximum Likelihood Estimates, Parameter Estimation, and analysis of Wald confidence interval, and the results are illustrated in TABLE 11.

TABLE 11

| | | | | | Pr > | | |
|---|---|---|---|---|---|---|---|
| | | | Standard | Wald | Chi- | 95% Confidence | |
| Parameter | DF | Estimate | Error | Chi-Square | Square | Limits | |
| Intercept | 1 | 2.4468 | 0.4155 | 34.6755 | <.0001 | 1.6324 | 3.2611 |
| ln (APC) | 1 | 0.1271 | 0.0425 | 8.9422 | 0.0028 | 0.0438 | 0.2105 |
| ln (COX2) | 1 | 0.2260 | 0.0449 | 25.3204 | <.0001 | 0.1380 | 0.3140 |
| ln (miR-203) | 1 | 0.1091 | 0.0418 | 6.8210 | 0.0090 | 0.0272 | 0.1910 |
| ln (RASSF1A) | 1 | 0.1288 | 0.0471 | 7.4929 | 0.0062 | 0.0366 | 0.2210 |

(3) Odds Ratio Estimates and Profile-Likelihood Confidence Intervals

Ln(APC), ln(COX2), ln(RASSF1A) and ln(miR-203) of the above-mentioned five groups are performed by Odds Ratio Estimates and Profile-Likelihood Confidence Intervals, and the results are illustrated as TABLE 12.

TABLE 12

| Effect | Unit | Estimate | 95% Confidence Limits | |
|---|---|---|---|---|
| ln (APC) | 1.0000 | 1.136 | 1.046 | 1.238 |
| ln (COX2) | 1.0000 | 1.254 | 1.152 | 1.376 |
| ln (miR-203) | 1.0000 | 1.115 | 1.028 | 1.213 |
| ln (RASSF1A) | 1.0000 | 1.137 | 1.039 | 1.252 |

According to Table 12, the odds ratio in the risk of suffering from HCC increases 13.6% when ln(APC) rises in per one unit, the odds ratio in the risk of suffering from HCC increases 25.4% when ln(COX2) rises in per one unit, the odds ratio in the risk of suffering from HCC increases 11.5% when ln(miRNA-203) rises in per one unit, and the odds ratio in the risk of suffering from HCC increases 13.7% when ln(RASSF1A) rises in per one unit, in which the degree of methylation in COX2 is the most influential among the four above-mentioned genes.

After foregoing analysis, stepwise regression analysis is performed to select the four variables ln(APC), ln(COX2), ln(miR-203) and ln(RASSF1A) to establish the model as follows.

Prediction model B: Ln(P/(1−P))=2.447+0.127×ln(APC)+0.226×In(COX2)+0.1091×ln(miR-203)+0.1288×ln(RASSF1A)

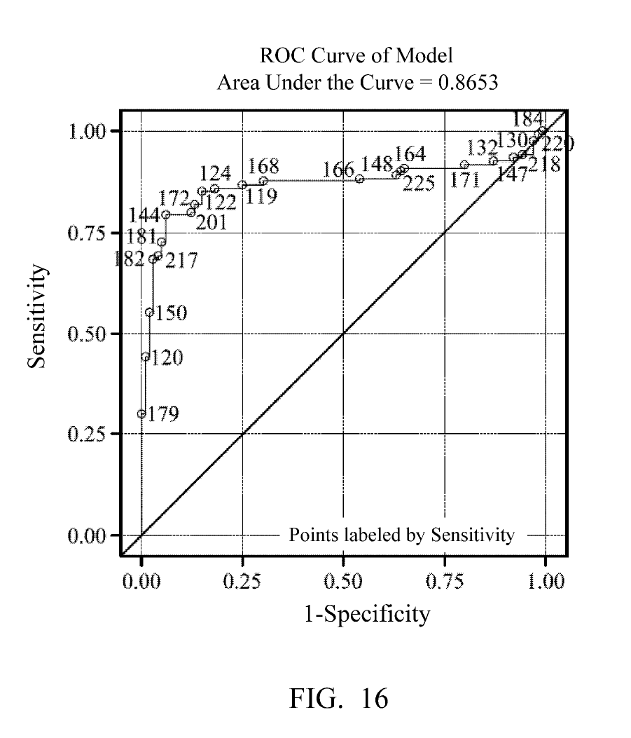

Then, ROC curve analysis is performed and the results are illustrated as TABLE 13 and FIG. 16.

TABLE 13
The relationship among cutoff value of ROC curve, sensitivity,
specificity, false positives, false negatives and overall accuracy

| | Correct | | Incorrect | | percentage | | | | |
|---|---|---|---|---|---|---|---|---|---|
| Cut-off | | Non- | | Non- | | | | False | False |
| Value | Event | event | Event | event | Correct | Sensitivity | Specificity | POS | NEG |
| 0.200 | 106 | 36 | 64 | 14 | 64.5 | 88.3 | 36.0 | 37.6 | 28.0 |
| 0.210 | 106 | 39 | 61 | 14 | 65.9 | 88.3 | 39.0 | 36.5 | 26.4 |
| 0.220 | 105 | 42 | 58 | 15 | 66.8 | 87.5 | 42.0 | 35.6 | 26.3 |
| 0.230 | 105 | 46 | 54 | 15 | 68.6 | 87.5 | 46.0 | 34.0 | 24.6 |
| 0.240 | 105 | 48 | 52 | 15 | 69.5 | 87.5 | 48.0 | 33.1 | 23.8 |
| 0.250 | 105 | 52 | 48 | 15 | 71.4 | 87.5 | 52.0 | 31.4 | 22.4 |
| 0.260 | 105 | 55 | 45 | 15 | 72.7 | 87.5 | 55.0 | 30.0 | 21.4 |
| 0.270 | 105 | 57 | 43 | 15 | 73.6 | 87.5 | 57.0 | 29.1 | 20.8 |
| 0.280 | 104 | 61 | 39 | 16 | 75.0 | 86.7 | 61.0 | 27.3 | 20.8 |
| 0.290 | 104 | 62 | 38 | 16 | 75.5 | 86.7 | 62.0 | 26.8 | 20.5 |
| 0.300 | 104 | 65 | 35 | 16 | 76.8 | 86.7 | 65.0 | 25.2 | 19.8 |
| 0.310 | 104 | 67 | 33 | 16 | 77.7 | 86.7 | 67.0 | 24.1 | 19.3 |
| 0.320 | 104 | 70 | 30 | 16 | 79.1 | 86.7 | 70.0 | 22.4 | 18.6 |
| 0.330 | 103 | 73 | 27 | 17 | 80.0 | 85.8 | 73.0 | 20.8 | 18.9 |
| 0.340 | 103 | 73 | 27 | 17 | 80.0 | 85.8 | 73.0 | 20.8 | 18.9 |
| 0.350 | 103 | 75 | 25 | 17 | 80.9 | 85.8 | 75.0 | 19.5 | 18.5 |
| 0.360 | 102 | 77 | 23 | 18 | 81.4 | 85.0 | 77.0 | 18.4 | 18.9 |
| 0.370 | 102 | 79 | 21 | 18 | 82.3 | 85.0 | 79.0 | 17.1 | 18.6 |
| 0.380 | 101 | 80 | 20 | 19 | 82.3 | 84.2 | 80.0 | 16.5 | 19.2 |
| 0.390 | 101 | 81 | 19 | 19 | 82.7 | 84.2 | 81.0 | 15.8 | 19.0 |
| 0.400 | 101 | 83 | 17 | 19 | 83.6 | 84.2 | 83.0 | 14.4 | 18.6 |
| 0.410 | 100 | 85 | 15 | 20 | 84.1 | 83.3 | 85.0 | 13.0 | 19.0 |
| 0.420 | 99 | 85 | 15 | 21 | 83.6 | 82.5 | 85.0 | 13.2 | 19.8 |
| 0.430 | 98 | 85 | 15 | 22 | 83.2 | 81.7 | 85.0 | 13.3 | 20.6 |
| 0.440 | 98 | 85 | 15 | 22 | 83.2 | 81.7 | 85.0 | 13.3 | 20.6 |
| 0.450 | 98 | 85 | 15 | 22 | 83.2 | 81.7 | 85.0 | 13.3 | 20.6 |
| 0.460 | 96 | 85 | 15 | 24 | 82.3 | 80.0 | 85.0 | 13.5 | 22.0 |
| 0.470 | 96 | 85 | 15 | 24 | 82.3 | 80.0 | 85.0 | 13.5 | 22.0 |
| 0.480 | 96 | 85 | 15 | 24 | 82.3 | 80.0 | 85.0 | 13.5 | 22.0 |
| 0.490 | 96 | 87 | 13 | 24 | 83.2 | 80.0 | 87.0 | 11.9 | 21.6 |
| 0.500 | 95 | 87 | 13 | 25 | 82.7 | 79.2 | 87.0 | 12.0 | 22.3 |
| 0.510 | 95 | 87 | 13 | 25 | 82.7 | 79.2 | 87.0 | 12.0 | 22.3 |
| 0.520 | 95 | 87 | 13 | 25 | 82.7 | 79.2 | 87.0 | 12.0 | 22.3 |
| 0.530 | 95 | 87 | 13 | 25 | 82.7 | 79.2 | 87.0 | 12.0 | 22.3 |
| 0.540 | 95 | 87 | 13 | 25 | 82.7 | 79.2 | 87.0 | 12.0 | 22.3 |
| 0.550 | 95 | 88 | 12 | 25 | 83.2 | 79.2 | 88.0 | 11.2 | 22.1 |
| 0.560 | 95 | 88 | 12 | 25 | 83.2 | 79.2 | 88.0 | 11.2 | 22.1 |
| 0.570 | 94 | 90 | 10 | 26 | 83.6 | 78.3 | 90.0 | 9.6 | 22.4 |
| 0.580 | 94 | 90 | 10 | 26 | 83.6 | 78.3 | 90.0 | 9.6 | 22.4 |
| 0.590 | 94 | 90 | 10 | 26 | 83.6 | 78.3 | 90.0 | 9.6 | 22.4 |
| 0.600 | 93 | 90 | 10 | 27 | 83.2 | 77.5 | 90.0 | 9.7 | 23.1 |
| 0.610 | 93 | 90 | 10 | 27 | 83.2 | 77.5 | 90.0 | 9.7 | 23.1 |
| 0.620 | 92 | 92 | 8 | 28 | 83.6 | 76.7 | 92.0 | 8.0 | 23.3 |
| 0.630 | 92 | 93 | 7 | 28 | 84.1 | 76.7 | 93.0 | 7.1 | 23.1 |
| 0.640 | 91 | 94 | 6 | 29 | 84.1 | 75.8 | 94.0 | 6.2 | 23.6 |
| 0.650 | 88 | 94 | 6 | 32 | 82.7 | 73.3 | 94.0 | 6.4 | 25.4 |
| 0.660 | 86 | 94 | 6 | 34 | 81.8 | 71.7 | 94.0 | 6.5 | 26.6 |
| 0.670 | 86 | 94 | 6 | 34 | 81.8 | 71.7 | 94.0 | 6.5 | 26.6 |
| 0.680 | 84 | 94 | 6 | 36 | 80.9 | 70.0 | 94.0 | 6.7 | 27.7 |
| 0.690 | 83 | 94 | 6 | 37 | 80.5 | 69.2 | 94.0 | 6.7 | 28.2 |
| 0.700 | 82 | 94 | 6 | 38 | 80.0 | 68.3 | 94.0 | 6.8 | 28.8 |
| 0.710 | 80 | 94 | 6 | 40 | 79.1 | 66.7 | 94.0 | 7.0 | 29.9 |
| 0.720 | 77 | 95 | 5 | 43 | 78.2 | 64.2 | 95.0 | 6.1 | 31.2 |
| 0.730 | 77 | 95 | 5 | 43 | 78.2 | 64.2 | 95.0 | 6.1 | 31.2 |
| 0.740 | 77 | 95 | 5 | 43 | 78.2 | 64.2 | 95.0 | 6.1 | 31.2 |
| 0.750 | 77 | 97 | 3 | 43 | 79.1 | 64.2 | 97.0 | 3.8 | 30.7 |
| 0.760 | 76 | 97 | 3 | 44 | 78.6 | 63.3 | 97.0 | 3.8 | 31.2 |
| 0.770 | 76 | 97 | 3 | 44 | 78.6 | 63.3 | 97.0 | 3.8 | 31.2 |
| 0.780 | 74 | 97 | 3 | 46 | 77.7 | 61.7 | 97.0 | 3.9 | 32.2 |
| 0.790 | 74 | 97 | 3 | 46 | 77.7 | 61.7 | 97.0 | 3.9 | 32.2 |
| 0.800 | 71 | 97 | 3 | 49 | 76.4 | 59.2 | 97.0 | 4.1 | 33.6 |

According to TABLE 13 and FIG. 16, area of ROC curve is 0.865, indicating that a better classification result is obtained by performing prediction model B to classify non-hepatitis B-related liver cancer groups and hepatitis B-related liver cancer groups. When the best cutoff value of model is 0.4, the sensitivity of 84.2%, specificity of 83.0%, false positives of 14.4%, false negatives of 18.6% as well as overall accuracy of 83.6% are obtained.

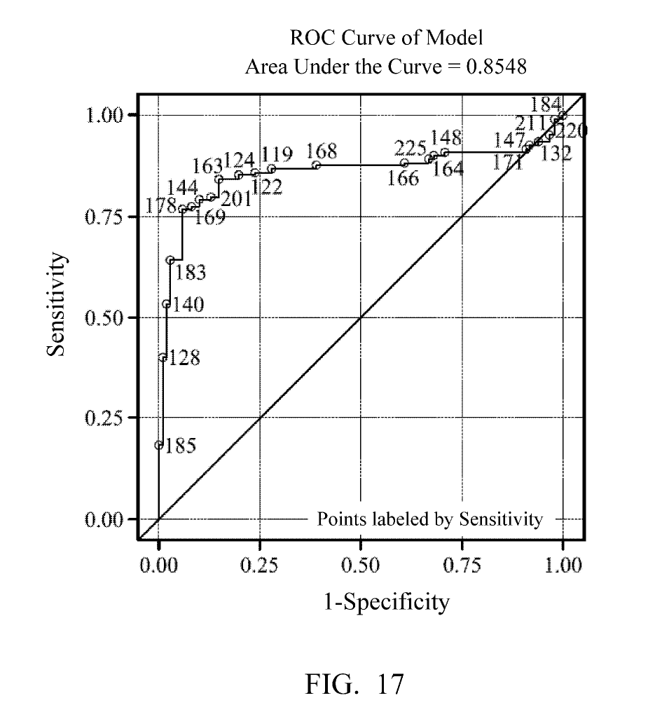

In addition, the method of Leave-one-out cross-validation (LOOCV) is performed to verify the model and used for confirming the classification capacity of this model. The results is illustrated in Table 14 and FIG. 17.

TABLE 14
The relationship among cutoff value, sensitivity, specificity, false
positives, false negatives and overall accuracy of ROC curve

| | Correct | | Incorrect | | percentage | | | | |
|---|---|---|---|---|---|---|---|---|---|
| Cut-off | | Non- | | Non- | | | | False | False |
| Value | Event | event | Event | event | Correct | Sensitivity | Specificity | POS | NEG |
| 0.200 | 106 | 36 | 64 | 14 | 64.5 | 88.3 | 36.0 | 37.6 | 28.0 |
| 0.210 | 106 | 39 | 61 | 14 | 65.9 | 88.3 | 39.0 | 36.5 | 26.4 |
| 0.220 | 105 | 42 | 58 | 15 | 66.8 | 87.5 | 42.0 | 35.6 | 26.3 |
| 0.230 | 105 | 46 | 54 | 15 | 68.6 | 87.5 | 46.0 | 34.0 | 24.6 |
| 0.240 | 105 | 48 | 52 | 15 | 69.5 | 87.5 | 48.0 | 33.1 | 23.8 |
| 0.250 | 105 | 52 | 48 | 15 | 71.4 | 87.5 | 52.0 | 31.4 | 22.4 |
| 0.260 | 105 | 55 | 45 | 15 | 72.7 | 87.5 | 55.0 | 30.0 | 21.4 |
| 0.270 | 105 | 57 | 43 | 15 | 73.6 | 87.5 | 57.0 | 29.1 | 20.8 |
| 0.280 | 104 | 61 | 39 | 16 | 75.0 | 86.7 | 61.0 | 27.3 | 20.8 |
| 0.290 | 104 | 62 | 38 | 16 | 75.5 | 86.7 | 62.0 | 26.8 | 20.5 |
| 0.300 | 104 | 65 | 35 | 16 | 76.8 | 86.7 | 65.0 | 25.2 | 19.8 |
| 0.310 | 104 | 67 | 33 | 16 | 77.7 | 86.7 | 67.0 | 24.1 | 19.3 |
| 0.320 | 104 | 70 | 30 | 16 | 79.1 | 86.7 | 70.0 | 22.4 | 18.6 |
| 0.330 | 103 | 73 | 27 | 17 | 80.0 | 85.8 | 73.0 | 20.8 | 18.9 |
| 0.340 | 103 | 73 | 27 | 17 | 80.0 | 85.8 | 73.0 | 20.8 | 18.9 |
| 0.350 | 103 | 75 | 25 | 17 | 80.9 | 85.8 | 75.0 | 19.5 | 18.5 |
| 0.360 | 102 | 77 | 23 | 18 | 81.4 | 85.0 | 77.0 | 18.4 | 18.9 |
| 0.370 | 102 | 79 | 21 | 18 | 82.3 | 85.0 | 79.0 | 17.1 | 18.6 |
| 0.380 | 101 | 80 | 20 | 19 | 82.3 | 84.2 | 80.0 | 16.5 | 19.2 |
| 0.390 | 101 | 81 | 19 | 19 | 82.7 | 84.2 | 81.0 | 15.8 | 19.0 |
| 0.400 | 101 | 83 | 17 | 19 | 83.6 | 84.2 | 83.0 | 14.4 | 18.6 |
| 0.410 | 100 | 85 | 15 | 20 | 84.1 | 83.3 | 85.0 | 13.0 | 19.0 |
| 0.420 | 99 | 85 | 15 | 21 | 83.6 | 82.5 | 85.0 | 13.2 | 19.8 |
| 0.430 | 98 | 85 | 15 | 22 | 83.2 | 81.7 | 85.0 | 13.3 | 20.6 |
| 0.440 | 98 | 85 | 15 | 22 | 83.2 | 81.7 | 85.0 | 13.3 | 20.6 |
| 0.450 | 98 | 85 | 15 | 22 | 83.2 | 81.7 | 85.0 | 13.3 | 20.6 |
| 0.460 | 96 | 85 | 15 | 24 | 82.3 | 80.0 | 85.0 | 13.5 | 22.0 |
| 0.470 | 96 | 85 | 15 | 24 | 82.3 | 80.0 | 85.0 | 13.5 | 22.0 |
| 0.480 | 96 | 85 | 15 | 24 | 82.3 | 80.0 | 85.0 | 13.5 | 22.0 |
| 0.490 | 96 | 87 | 13 | 24 | 83.2 | 80.0 | 87.0 | 11.9 | 21.6 |
| 0.500 | 95 | 87 | 13 | 25 | 82.7 | 79.2 | 87.0 | 12.0 | 22.3 |
| 0.510 | 95 | 87 | 13 | 25 | 82.7 | 79.2 | 87.0 | 12.0 | 22.3 |
| 0.520 | 95 | 87 | 13 | 25 | 82.7 | 79.2 | 87.0 | 12.0 | 22.3 |
| 0.530 | 95 | 87 | 13 | 25 | 82.7 | 79.2 | 87.0 | 12.0 | 22.3 |
| 0.540 | 95 | 87 | 13 | 25 | 82.7 | 79.2 | 87.0 | 12.0 | 22.3 |
| 0.550 | 95 | 88 | 12 | 25 | 83.2 | 79.2 | 88.0 | 11.2 | 22.1 |
| 0.560 | 95 | 88 | 12 | 25 | 83.2 | 79.2 | 88.0 | 11.2 | 22.1 |
| 0.570 | 94 | 90 | 10 | 26 | 83.6 | 78.3 | 90.0 | 9.6 | 22.4 |
| 0.580 | 94 | 90 | 10 | 26 | 83.6 | 78.3 | 90.0 | 9.6 | 22.4 |
| 0.590 | 94 | 90 | 10 | 26 | 83.6 | 78.3 | 90.0 | 9.6 | 22.4 |
| 0.600 | 93 | 90 | 10 | 27 | 83.2 | 77.5 | 90.0 | 9.7 | 23.1 |
| 0.610 | 93 | 90 | 10 | 27 | 83.2 | 77.5 | 90.0 | 9.7 | 23.1 |
| 0.620 | 92 | 92 | 8 | 28 | 83.6 | 76.7 | 92.0 | 8.0 | 23.3 |
| 0.630 | 92 | 93 | 7 | 28 | 84.1 | 76.7 | 93.0 | 7.1 | 23.1 |
| 0.640 | 91 | 94 | 6 | 29 | 84.1 | 75.8 | 94.0 | 6.2 | 23.6 |
| 0.650 | 88 | 94 | 6 | 32 | 82.7 | 73.3 | 94.0 | 6.4 | 25.4 |
| 0.660 | 86 | 94 | 6 | 34 | 81.8 | 71.7 | 94.0 | 6.5 | 26.6 |
| 0.670 | 86 | 94 | 6 | 34 | 81.8 | 71.7 | 94.0 | 6.5 | 26.6 |
| 0.680 | 84 | 94 | 6 | 36 | 80.9 | 70.0 | 94.0 | 6.7 | 27.7 |
| 0.690 | 83 | 94 | 6 | 37 | 80.5 | 69.2 | 94.0 | 6.7 | 28.2 |
| 0.700 | 82 | 94 | 6 | 38 | 80.0 | 68.3 | 94.0 | 6.8 | 28.8 |
| 0.710 | 80 | 94 | 6 | 40 | 79.1 | 66.7 | 94.0 | 7.0 | 29.9 |
| 0.720 | 77 | 95 | 5 | 43 | 78.2 | 64.2 | 95.0 | 6.1 | 31.2 |
| 0.730 | 77 | 95 | 5 | 43 | 78.2 | 64.2 | 95.0 | 6.1 | 31.2 |
| 0.740 | 77 | 95 | 5 | 43 | 78.2 | 64.2 | 95.0 | 6.1 | 31.2 |
| 0.750 | 77 | 97 | 3 | 43 | 79.1 | 64.2 | 97.0 | 3.8 | 30.7 |
| 0.760 | 76 | 97 | 3 | 44 | 78.6 | 63.3 | 97.0 | 3.8 | 31.2 |
| 0.770 | 76 | 97 | 3 | 44 | 78.6 | 63.3 | 97.0 | 3.8 | 31.2 |
| 0.780 | 74 | 97 | 3 | 46 | 77.7 | 61.7 | 97.0 | 3.9 | 32.2 |
| 0.790 | 74 | 97 | 3 | 46 | 77.7 | 61.7 | 97.0 | 3.9 | 32.2 |
| 0.800 | 71 | 97 | 3 | 49 | 76.4 | 59.2 | 97.0 | 4.1 | 33.6 |

According to TABLE 13, 14 and FIG. 17, when the method of Leave-one-out cross-validation (LOOCV) is performed to verify the model, the area of ROC curve is 0.8548. When the best cutoff value of model is 0.4, the obtained sensitivity, specificity, false positives, false negatives as well as overall accuracy are the same with the original model, certifying the accuracy of the prediction mode.

3. AFP Marker for Prediction of Hepatitis B-Related Liver Cancer

Ln(AFP) of the above-mentioned five groups is performed to establish the model as follows.

Prediction model: ln(P/(1−P))=0.8159+0.1685×ln(AFP)

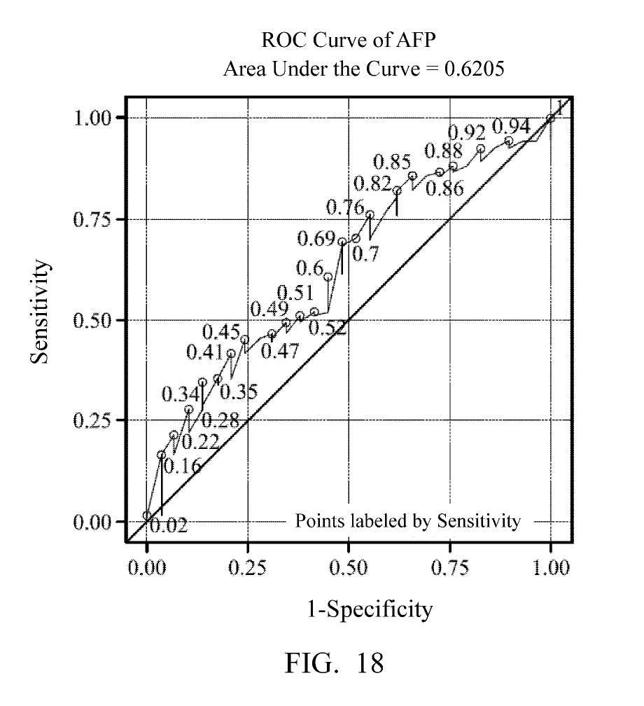

Then, ROC curve analysis is performed and the results are illustrated in TABLE 15 and FIG. 18. When the best cutoff value of model is 0.775, the corresponding AFP (ng/ml) value is 12.1545, sensitivity is 50.9%, specificity is 62.1%, false positives is 15.7%, false negatives is 76% as well as overall accuracy is 53.1%.

TABLE 15

| | AFP (ng/ml) | | | Sensitivity | 50.9 |
|---|---|---|---|---|---|
| | >12.1545 | ≦12.1545 | Total | Specificity | 62.1 |
| HCC Cases | 59 | 57 | 116 | False POS | 15.7 |
| Non-HCC Cases | 11 | 18 | 29 | False NEG | 76.0 |
| Total | 70 | 75 | 145 | Correct | 53.1 |

According to the above-mentioned results, the prediction model of combination of ln(APC), ln(COX2), ln(RASSF1A) and ln(miR-203) has the highest accuracy.

D. Survival Analysis

(1) Univariate Survival Analysis

The patients suffering from liver cancer are grouped based on age, gender, AFP value, level of vascular invasion, tumor size, clinical stage, suffering from hepatitis virus or not, suffering from cirrhosis or not and the predicted score A higher than 0.45 or not. Then, univariate analysis of death in 5 years is performed and the result is illustrated in TABLE 16.

TABLE 16
Univariate analysis of death in 5 years

| | | Death | | |
|---|---|---|---|---|
| Parameters | DF | No. | percentage | P-value |
| Gender | | | | 0.5771 |
| women | 42 | 12 | 28.57 | |
| Men | 138 | 49 | 35.51 | |
| Cirrhosis | | | | 0.0086 |
| Yes | 72 | 34 | 47.22 | |
| No | 104 | 25 | 24.04 | |
| Histologic grade | | | | 0.0382 |
| M (includes M to P) | 98 | 27 | 27.55 | |
| P | 13 | 5 | 38.46 | |
| W | 26 | 2 | 7.69 | |
| Tumor size (cm) | | | | 0.3251 |
| ≦5 | 133 | 42 | 31.58 | |
| >5 | 47 | 19 | 40.43 | |
| AFP (ng/ml) | | | | <.0001 |
| ≦20 | 96 | 20 | 20.83 | |
| >20 | 84 | 41 | 48.81 | |
| Pathological stage | | | | 0.0054 |
| stage I, II | 161 | 49 | 30.43 | |
| stage III, IV | 19 | 12 | 64.16 | |
| Clinical Stage | | | | <.0001 |
| stage I, II | 143 | 35 | 24.48 | |
| stage III, IV | 37 | 26 | 70.27 | |
| Vascular Invasion | | | | <.0001 |
| Yes | 130 | 30 | 23.08 | |
| No | 41 | 25 | 60.98 | |
| Predicted score A of HCC | | | | 0.0052 |
| ≦0.45 | 50 | 8 | 16.00 | |
| >0.45 | 130 | 53 | 40.77 | |

NOTE:
Prediction Score A of HCC is obtained by the following formula:
Prediction Score A = exp(predicted value A)/(1 + exp (predicted value A))
Predictive value A = 2.238 + 0.0898 × ln (APC) + 0.1875 × ln (COX2) + 0.0701 × ln (miR-203) + 0.1097 × ln (RASSFIA)

According to Table 16, five-year death rate of each group is listed and the survival function is tested by Log-rank Test. As illustrated above, regarding the seven variables including cirrhosis, histologic grade, AFP (ng/ml), pathological stage, clinical stage, vascular invasion and predicted score A, the survival function of these variables are significantly different from each other.

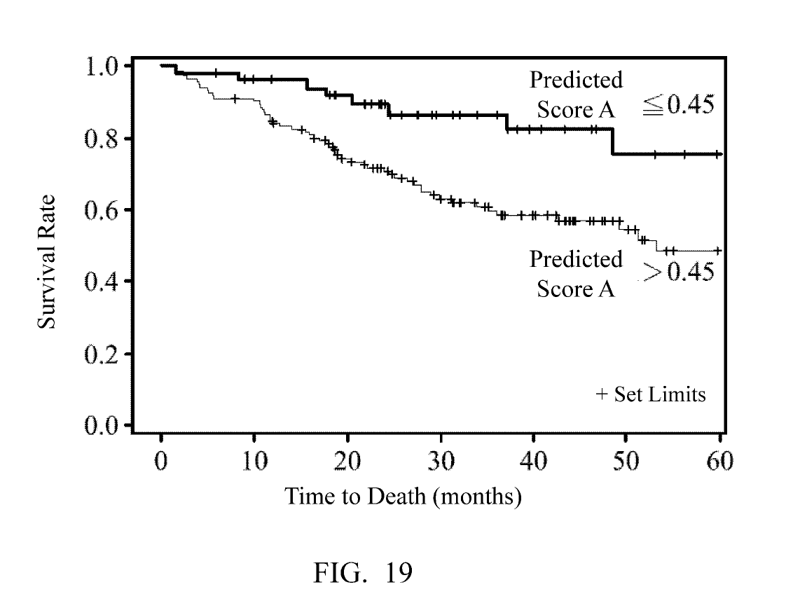

Five-year univariate survival analysis for predicted score A of HCC is illustrated in FIG. 19. When the prediction score A is less than or equal to 0.45, the 5-year survival probability is 75.2%. When the prediction score A is greater than 0.45, the 5-year survival probability is 48.3%, in which P value is 0.0052, indicating that a significant difference is existed.

(2) Multivariable Survival Analysis

The patients suffering from liver cancer are grouped based on age, gender, AFP value, level of vascular invasion, tumor size, clinical stage, suffering from hepatitis virus or not, suffering from cirrhosis or not and the prediction score A higher than 0.45 or not. Then, multivariate survival analysis is performed.

Multivariate Cox Proportional Hazard Regression Analysis

The patients suffering from liver cancer grouped as above-mentioned classification are analyzed by Multivariate Cox Proportional Hazard Regression and the result is illustrated in TABLE 17.

TABLE 17

| | | | Standard | Chi- | Pr > Chi- | Hazard | 95% Confidence | |
|---|---|---|---|---|---|---|---|---|
| Parameters | DF | Estimate | Error | Square | Square | ration | Limits | |
| Age | 1 | 0.01398 | 0.01515 | 0.8509 | 0.3563 | 1.014 | 0.984 | 1.045 |
| Gender (Men vs. | 1 | −0.04761 | 0.40734 | 0.0137 | 0.9070 | 0.954 | 0.429 | 2.119 |
| women) | | | | | | | | |
| AFP (<=20 | 1 | 0.69494 | 0.30271 | 5.2704 | 0.0217 | 2.004 | 1.107 | 3.626 |
| vs. >20) | | | | | | | | |
| Vascular Invasion | 1 | 0.50467 | 0.30069 | 2.8169 | 0.0933 | 1.656 | 0.919 | 2.986 |
| (Yes or No) | | | | | | | | |
| Tumor size (<=5 cm | 1 | −0.18205 | 0.35056 | 0.2697 | 0.6035 | 0.834 | 0.419 | 1.657 |
| vs. >5 cm) | | | | | | | | |
| Clinical Stage | 1 | 1.47360 | 0.34281 | 18.4784 | <.0001 | 4.365 | 2.229 | 8.546 |
| (III/IV vs. I/II) | | | | | | | | |
| Fibrosis (Yes or | 1 | 0.69139 | 0.31223 | 4.9033 | 0.0268 | 1.996 | 1.083 | 3.682 |
| No) | | | | | | | | |
| Prediction score A | 1 | 1.08088 | 0.45347 | 5.6814 | 0.0171 | 2.947 | 1.212 | 7.168 |
| of HCC (>0.45 vs. | | | | | | | | |
| 0.45) | | | | | | | | |

Multivariate Cox Proportional Hazard Regression Analysis is performed to analyze multivariate survival functions, in which AFP, clinical stage and prediction score A remain statistically significant while adjusting the other variables. If the AFP value of the subject is higher than 20, the risk ratio of 5-year death is increased by 1.0 times. If the clinical stage of the subject is classified as more than third, the risk ratio of 5-year death is increased by about 3.4 times. If the prediction score A of the subject is greater than 0.45, the risk ratio of 5-year death is increased by about 1.9 times.

The formula obtained from Multivariate Cox Proportional Hazard Regression Analysis is as follows:

Predicted score=B₁×(age)+B₂×(gender)+B₃×(AFP value higher than 20 or not)+B₄×(level of vascular invasion)+B₅×(tumor size higher than 5 cm or not)+B₆×(clinical stage)+B₇×(suffering from cirrhosis or not)+B₈×(predicted score A higher than 0.45 or not), in which B₁ is 0.01398, B₂ is −0.04761, B₃ is 0.69494, B₄ is 0.50467, B₅ is −0.18205, B₆ is 1.47360, B₇ is 0.69139, and B₈ is 1.08088.

In addition, age substitutes actual age, gender substitutes 1 for men and 0 for women, AFP value higher than 20 or not substitutes 1 for yes and 0 for no, level of vascular invasion substitutes 1 for yes and 0 for no, tumor size higher than 5 cm or not substitutes 1 for yes and 0 for no, clinical stage substitutes 1 for III/IV and 0 for I/II, suffering from cirrhosis or not substitutes 1 for yes and 0 for no, and predicted score A higher than 0.45 or not substitutes 1 for yes and 0 for no.

Breslow method is performed to predict the survival probability in the estimated survival time t(year) by the formula: survival probability in estimated survival time t(year)=(S0(t))exp(prediction score), in which S0(t) is survival probability in t year. The function of survival probability in t year S0(t) is illustrated in Table 18. In addition, the above-mentioned function of survival probability in t year S0(t) is calculated by referring to the literatures: Breslow, N. (1974) Covariance Analysis of Survival Data under the Proportional Hazards Model. International Statistical Review, 43, 43-54; and Elisa, T. Lee and John Wenyu Wang. (2003) Statistical Methods for Survival Data Analysis. P. 321. 3rd ed. Wiley, N.Y.

TABLE 18
Survival probability in t year S0(t)

| Time (month) | S0(t) | Lower Limit | Upper Limit |
|---|---|---|---|
| 0 | 1 | | |
| 1.4 | 0.99975 | 0.998955 | 1 |
| 1.466667 | 0.999497 | 0.998059 | 1 |
| 1.733333 | 0.999238 | 0.997149 | 1 |
| 2.333333 | 0.99869 | 0.995241 | 1 |
| 2.7 | 0.998412 | 0.994276 | 1 |
| 4.033333 | 0.998125 | 0.993286 | 1 |
| 4.166667 | 0.997831 | 0.99227 | 1 |
| 5 | 0.997519 | 0.991199 | 1 |
| 5.566667 | 0.997202 | 0.990113 | 1 |
| 5.666667 | 0.996881 | 0.989014 | 1 |
| 9.933333 | 0.996553 | 0.98789 | 1 |
| 10.6 | 0.996223 | 0.986765 | 1 |
| 10.7 | 0.995891 | 0.985632 | 1 |
| 10.86667 | 0.995553 | 0.98448 | 1 |
| 11.1 | 0.995207 | 0.983305 | 1 |
| 11.16667 | 0.994853 | 0.982103 | 1 |
| 11.7 | 0.994495 | 0.980891 | 1 |
| 11.76667 | 0.994134 | 0.97967 | 1 |
| 12.06667 | 0.993747 | 0.978363 | 1 |
| 12.83333 | 0.993356 | 0.977047 | 1 |
| 14.03333 | 0.992958 | 0.9757 | 1 |
| 15.96667 | 0.992541 | 0.974291 | 1 |
| 16.46667 | 0.992122 | 0.972877 | 1 |
| 17.1 | 0.991697 | 0.971444 | 1 |
| 17.76667 | 0.99127 | 0.970002 | 1 |
| 17.83333 | 0.990841 | 0.96856 | 1 |
| 18.3 | 0.990396 | 0.967064 | 1 |
| 18.5 | 0.989944 | 0.965541 | 1 |
| 18.7 | 0.98948 | 0.963985 | 1 |
| 18.73333 | 0.989013 | 0.962419 | 1 |
| 18.96667 | 0.988536 | 0.960818 | 1 |
| 20.53333 | 0.988022 | 0.959096 | 1 |
| 20.56667 | 0.987507 | 0.95737 | 1 |
| 21.83333 | 0.986982 | 0.955615 | 1 |
| 22.33333 | 0.986447 | 0.953825 | 1 |
| 24 | 0.985893 | 0.951975 | 1 |
| 24.56667 | 0.985298 | 0.949997 | 1 |
| 24.63333 | 0.9847 | 0.948011 | 1 |
| 25.16667 | 0.984087 | 0.945975 | 1 |
| 26.6 | 0.983452 | 0.943871 | 1 |
| 28.03333 | 0.982802 | 0.941715 | 1 |
| 28.1 | 0.982072 | 0.939301 | 1 |
| 29.06667 | 0.981335 | 0.936864 | 1 |
| 29.96667 | 0.980573 | 0.934361 | 1 |
| 34.06667 | 0.979729 | 0.931583 | 1 |
| 35.36667 | 0.978851 | 0.928695 | 1 |
| 36.2 | 0.977944 | 0.925717 | 1 |
| 37.3 | 0.976973 | 0.922537 | 1 |
| 42.83333 | 0.97578 | 0.918602 | 1 |
| 48.86667 | 0.974163 | 0.913205 | 1 |
| 51.43333 | 0.972426 | 0.907406 | 1 |
| 53.36667 | 0.969968 | 0.899283 | 1 |
| 60 | 0.969968 | 0.899283 | 1 |

(a) Prediction of the Survival Probability by Prediction Scores A of HCC

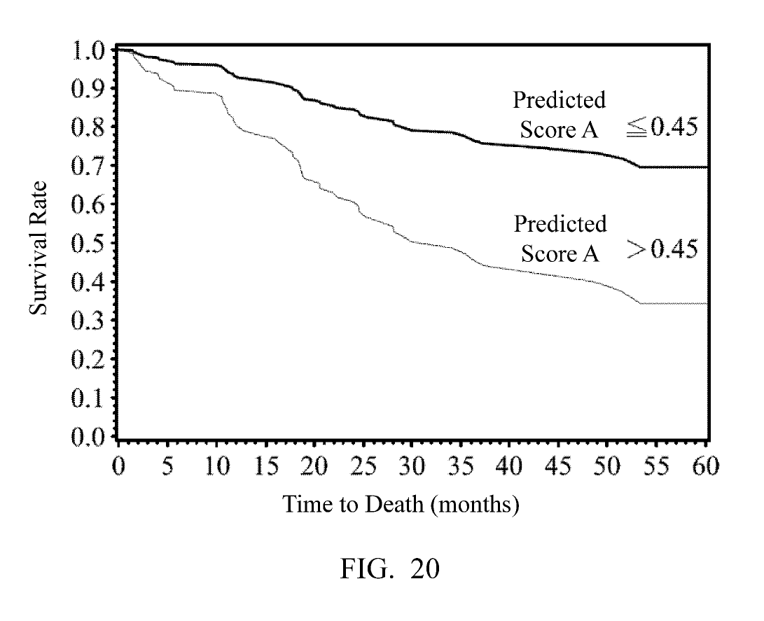

In case of prediction scores A of HCC, after being adjusted by median of other variables composite value and estimated by Breslow method to adjust the Covariate-Adjusted Survival Function, the differences in survival function between two portfolio groups of prediction score A of HCC are illustrated in FIG. 20.

As illustrated in FIG. 20, while the prediction score A of the subject with liver cancer is less than or equal to 0.45, the five-year survival probability is approximately 69.48%, and while the prediction score A of the subject with liver cancer is greater than 0.45, the five-year survival probability is approximately 34.19%.

(b) Prediction of the Survival Probability by Prediction Ccores A and AFP Value

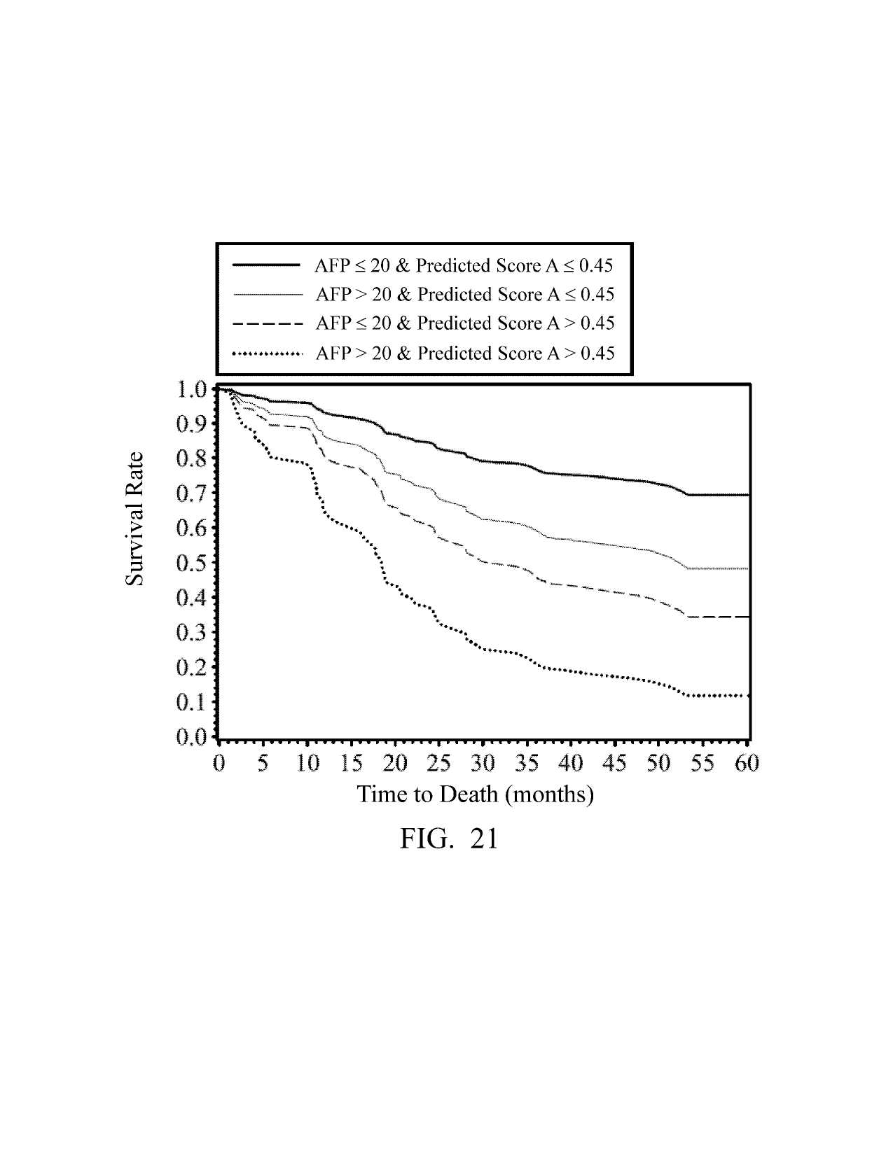

In case of prediction scores A and AFP value, after being adjusted by median of composite value in other variables, and estimated and adjusted by Breslow method to adjust the Covariate-Adjusted Survival Function, the differences in survival function among four portfolio groups of prediction score A and AFP value are illustrated in FIG. 21.

As illustrated in FIG. 21, when the subject suffering from liver cancer is detected with AFP value of less than or equal to 20 (ng/ml) and prediction score A of less than or equal to 0.45, the five-year survival probability is 69.48%. When the subject suffering from liver cancer is detected with AFP value of greater than 20 (ng/ml) and prediction score A of less than or equal to 0.45, the five-year survival probability is 48.61%. When the subject suffering from liver cancer is detected with AFP value of less than or equal to 20 (ng/ml) and prediction score A of greater than 0.45, the five-year survival probability is 34.19%. When the subject suffering from liver cancer is detected with AFP value of greater than 20 (ng/ml) and prediction score A of greater than 0.45, the five-year survival probability is remaining 11.64%.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A method for evaluating a risk of liver cancer in a subject, comprising the following steps of (a) to (c):
(a) detecting methylation levels of APC gene, COX2 gene, RASSF1A gene and miR-203 gene respectively in a bio-sample from the subject;
(b) calculating a predicted score A according to the methylation levels of APC gene, COX2 gene, RASSF1A gene and miR-203 gene; and
(c) evaluating the risk level of liver cancer in the subject according to the predicted score A, wherein the predicted score A is higher relative to a pre-confirmed reference value, indicating that the subject has the risk of afflicting with liver cancer.

2. The method according to claim 1, wherein the pre-confirmed reference value is determined by comparing the methylation levels of APC gene, COX2 gene, RASSF1A gene and miR-203 gene in one group of subjects known to non-live cancer with another group known to live cancer and obtaining a cutoff value according to the receiver operating characteristic (ROC) curve.

3. The method according to claim 1, wherein the predicted score A is based on the methylation levels of APC gene, COX2 gene, RASSF1A gene and miR-203 gene and calculated by the following formula:
predicted score A=exp(predicted value A)/(1+exp (predicted value A)), wherein the predicted value A=X₁+X₂×ln(APC)+X₃×ln(COX2)+X₄×ln(miR-203)+X₅×ln(RASSF1A);
wherein X₁ ranges from 1.6148 to 2.8618, X₂ ranges from 0.0237 to 0.1559, X₃ ranges from 0.1169 to 0.2581, X₄ ranges from 0.0058 to 0.1344, and X₅ ranges from 0.0436 to 0.1758; and
wherein ln(APC) represents a hyperbolic logarithm of the methylation level of APC gene, and the methylation level of APC gene is calculated from the following formula: 2̂(Ct(β-actin)−Ct(APC))×1000, ln(COX2) represents a hyperbolic logarithm of the methylation level of COX2 gene, and the methylation level of COX2 gene is calculated from the following formula: 2̂(Ct(β-actin)−Ct(COX2))×1000, ln(miR-203) represents a hyperbolic logarithm of the methylation level of miR-203 gene, and the methylation level of miR-203 gene is calculated from the following formula: 2̂(Ct(β-actin)−Ct(miR-203))×1000, and ln(RASSF1A) represents a hyperbolic logarithm of the methylation level of RASSF1A gene, and the methylation level of RASSF1A gene is calculated from the following formula: 2̂(Ct(β-actin)−Ct(RASSF1A))×1000.

4. The method according to claim 1, wherein the methylation level of miR-203 gene is detected by combining a primer-pair, a first probe and/or a second probe; and
wherein the primer-pair includes a sense primer and an antisense primer, the sense primer has a sequence of at least 85% sequence similarity to SEQ IDNO: 2, the antisense primer has a sequence of at least 85% sequence similarity to SEQ IDNO: 3, the first probe has a sequence of at least 85% sequence similarity to SEQ IDNO: 4, and the second probe has a sequence of at least 85% sequence similarity to SEQ IDNO: 5.

5. A method of preparing a kit for APC gene, COX2 gene, RASSF1A gene and miR-203 gene for evaluating a risk of liver cancer in a subject, comprising the following steps of (a) to (c):
(a) detecting methylation levels of APC gene, COX2 gene, RASSF1A gene and miR-203 gene respectively in a bio-sample from the subject;
(b) calculating a predicted score A according to the methylation levels of APC gene, COX2 gene, RASSF1A gene and miR-203 gene; and
(c) evaluating the risk level of liver cancer in the subject according to the predicted score A, wherein the predicted score A is higher relative to a pre-confirmed reference value, indicating that the subject has the risk of afflicting with liver cancer.

6. A method for evaluating a risk of afflicting with hepatitis B-related liver cancer in a subject infected with hepatitis B virus, comprising the following steps of (a) to (c):
(a) detecting methylation levels of APC gene, COX2 gene, RASSF1A gene and miR-203 gene respectively in a bio-sample from the subject infected with hepatitis B virus;
(b) calculating a predicted score B according to the methylation levels of APC gene, COX2 gene, RASSF1A gene and miR-203 gene; and
(c) evaluating the risk level of afflicting with hepatitis B-related liver cancer in the subject infected with hepatitis B virus according to the predicted score B, wherein the predicted score B is higher relative to a pre-confirmed reference value, indicating that the subject infected with hepatitis B virus has the risk of afflicting with hepatitis B virus-related liver cancer.

7. The method according to claim 6, wherein the pre-confirmed reference value is determined by comparing the methylation levels of APC gene, COX2 gene, RASSF1A gene and miR-203 gene in one group of subjects known to non-hepatitis B virus related live cancer with another group known to hepatitis B virus related live cancer and obtaining a cutoff value according to the receiver operating characteristic (ROC) curve.

8. The method according to claim 6, wherein the predicted score B is based on the methylation levels of APC gene, COX2 gene, RASSF1A gene and miR-203 gene and calculated by the following formula:
predicted score B=exp (predicted value B)/(1+exp(predicted value B)), wherein the predicted value B=Y₁+Y₂×ln(APC)+Y₃×ln(COX2)+Y₄×ln(miR-203)+Y₅×ln(RASSF1A);
wherein Y₁ ranges from 1.7 to 3.34, Y₂ ranges from 0.045 to 0.213, Y₃ ranges from 0.142 to 0.32, Y₄ ranges from 0.028 to 0.193, and Y₅ ranges from 0.038 to 0.224; and
wherein ln(APC) represents a hyperbolic logarithm of the methylation level of APC gene, and the methylation level of APC gene is calculated from the following formula: 2̂(Ct(β-actin)−Ct(APC))×1000, ln(COX2) represents a hyperbolic logarithm of the methylation level of COX2 gene, and the methylation level of COX2 gene is calculated from the following formula: 2̂(Ct(β-actin)−Ct(COX2))×1000, ln(miR-203) represents a hyperbolic logarithm of the methylation level of miR-203 gene, and the methylation level of miR-203 gene is calculated from the following formula: 2̂(Ct(β-actin)−Ct(miR-203))×1000, and ln(RASSF1A) represents a hyperbolic logarithm of the methylation level of RASSF1A gene, and the methylation level of RASSF1A gene is calculated from the following formula: 2̂(Ct(β-actin)−Ct(RASSF1A))×1000.

9. The method according to claim 1, wherein the methylation level of miR-203 gene is detected by combining a primer-pair, a first probe and/or a second probe; and
wherein the primer-pair includes a sense primer and an antisense primer, the sense primer has a sequence of at least 85% sequence similarity to SEQ IDNO: 2, the antisense primer has a sequence of at least 85% sequence similarity to SEQ IDNO: 3, the first probe has a sequence of at least 85% sequence similarity to SEQ IDNO: 4, and the second probe has a sequence of at least 85% sequence similarity to SEQ IDNO: 5.

10. A method of preparing a kit for APC gene, COX2 gene, RASSF1A gene and miR-203 gene for evaluating a risk of afflicting with hepatitis B-related liver cancer in a subject infected with hepatitis B virus, comprising the following steps of (a) to (c):
(a) detecting methylation levels of APC gene, COX2 gene, RASSF1A gene and miR-203 gene respectively in a bio-sample from the subject infected with hepatitis B virus;
(b) calculating a predicted score B according to the methylation levels of APC gene, COX2 gene, RASSF1A gene and miR-203 gene; and
(c) evaluating the risk level of liver cancer in the subject according to the predicted score B, wherein the predicted score B is higher relative to a pre-confirmed reference value, indicating that the subject infected with hepatitis B virus has the risk of afflicting with liver cancer.

11. A method for evaluating a prognosis of a subject afflicted with liver cancer, comprising the following steps of (a) to (c):
(a) detecting methylation levels of APC gene, COX2 gene, RASSF1A gene and miR-203 gene respectively in a bio-sample from the subject afflicted with liver cancer;
(b) calculating a predicted score A according to the methylation levels of APC gene, COX2 gene, RASSF1A gene and miR-203 gene; and
(c) evaluating a five-year survival probability of the subject afflicted with liver cancer according to the predicted score A.

12. The method according to claim 11, wherein the predicted score A is based on the methylation levels of APC gene, COX2 gene, RASSF1A gene and miR-203 gene and calculated by the following formula:
predicted score A=exp(predicted value A)/(1+exp(predicted value A)), wherein the predicted value A=X₁+X₂×ln(APC)+X₃×ln(COX2)+X₄×ln(miR-203)+X₅×ln(RASSF1A);
wherein X₁ ranges from 1.6148 to 2.8618, X₂ ranges from 0.0237 to 0.1559, X₃ ranges from 0.1169 to 0.2581, X₄ ranges from 0.0058 to 0.1344, and X₅ ranges from 0.0436 to 0.1758; and
wherein ln(APC) represents a hyperbolic logarithm of the methylation level of APC gene, and the methylation level of APC gene is calculated from the following formula: 2̂(Ct(β-actin)−Ct(APC))×1000, ln(COX2) represents a hyperbolic logarithm of the methylation level of COX2 gene, and the methylation level of COX2 gene is calculated from the following formula: 2̂(Ct(β-actin)−Ct(COX2))×1000, ln(miR-203) represents a hyperbolic logarithm of the methylation level of miR-203 gene, and the methylation level of miR-203 gene is calculated from the following formula: 2̂(Ct(β-actin)−Ct(miR-203))×1000, and ln(RASSF1A) represents a hyperbolic logarithm of the methylation level of RASSF1A gene, and the methylation level of RASSF1A gene is calculated from the following formula: 2̂(Ct(β-actin)−Ct(RASSF1A))×1000.

13. A method for evaluating a prognosis of a subject afflicted with liver cancer, comprising the following steps of (a) to (c):
(a) detecting methylation levels of APC gene, COX2 gene, RASSF1A gene and miR-203 gene respectively in a bio-sample from the subject afflicted with liver cancer;
(b) calculating a predicted score according to the methylation levels of APC gene, COX2 gene, RASSF1A gene and miR-203 gene, and age, gender, AFP value, level of vascular invasion, tumor size, clinical stage, suffering from hepatitis virus or not and suffering from cirrhosis or not; and
(c) evaluating a survival probability in a estimated survival time t (year) of the subject afflicted with liver cancer according to the predicted score.

14. The method according to claim 13, wherein the survival probability in the estimated survival time t(year) is calculated by the following formula:
survival probability in estimated survival time t(year)=(S0(t)exp(prediction score);
wherein S0(t) is survival probability in t year.

15. The method according to claim 13, wherein the step (b) comprises:
(i) calculating a predicted score A according to the methylation levels of APC gene, COX2 gene, RASSF1A gene and miR-203 gene; and
(ii) calculating a predicted score by combining the predicted score A with age, gender, AFP value, level of vascular invasion, tumor size, clinical stage, suffering from hepatitis virus or not and suffering from cirrhosis or not.

16. The method according to claim 15, wherein the predicted score A is based on the methylation levels of APC gene, COX2 gene, RASSF1A gene and miR-203 gene and calculated by the following formula:
predicted score A=exp (predicted value A)/(1+exp(predicted value A)), wherein the predicted value A=X₁+X₂×ln(APC)+X₃×ln(COX2)+X₄×ln(miR-203)+X₅ ×ln(RASSF1A);
wherein X₁ ranges from 1.6148 to 2.8618, X₂ ranges from 0.0237 to 0.1559, X₃ ranges from 0.1169 to 0.2581, X₄ ranges from 0.0058 to 0.1344, and X₅ ranges from 0.0436 to 0.1758; and
wherein ln(APC) represents a hyperbolic logarithm of the methylation level of APC gene, and the methylation level of APC gene is calculated from the following formula: 2̂(Ct(β-actin)−Ct(APC))×1000, ln(COX2) represents a hyperbolic logarithm of the methylation level of COX2 gene, and the methylation level of COX2 gene is calculated from the following formula: 2̂(Ct(β-actin)−Ct(COX2))×1000, ln(miR-203) represents a hyperbolic logarithm of the methylation level of miR-203 gene, and the methylation level of miR-203 gene is calculated from the following formula: 2̂(Ct(β-actin)−Ct(miR-203))×1000, and ln(RASSF1A) represents a hyperbolic logarithm of the methylation level of RASSF1A gene, and the methylation level of RASSF1A gene is calculated from the following formula: 2̂(Ct(β-actin)−Ct(RASSF1A))×1000.

17. The method according to claim 16, wherein the predicted score is based on age, gender, AFP value, level of vascular invasion, tumor size, clinical stage, suffering from hepatitis virus or not, suffering from cirrhosis or not and the predicted score A higher relative to a pre-confirmed reference value or not, and calculated by the following formula:
predicted score=B₁×(age)+B₂×(gender)+B₃×(AFP value higher than 20 or not)+B₄×(level of vascular invasion)+B₅×(tumor size higher than 5 cm or not)+B₆×(clinical stage)+B₇×(suffering from cirrhosis or not)+B₈×(predicted score A higher relative to a pre-confirmed reference value or not);
wherein B₁ ranges from −0.0224 to 0.0426, B₂ ranges from −0.8233 to 0.7836, B₃ ranges from 0.1798 to 1.3902, B₄ ranges from −0.1089 to 1.0898, B₅ ranges from −0.9560 to 0.4118, B₆ ranges from 0.8525 to 2.202, B₇ ranges from -1.9221 to −0.2812, and B₈ ranges from 0.3534 to 2.2217; and
wherein age substitutes actual age, gender substitutes 1 for men and 0 for women, AFP value higher than 20 or not substitutes 1 for yes and 0 for no, level of vascular invasion substitutes 1 for yes and 0 for no, tumor size higher than 5 cm or not substitutes 1 for yes and 0 for no, clinical stage substitutes 1 for III/IV and 0 for I/II, suffering from cirrhosis or not substitutes 1 for yes and 0 for no, and predicted score A higher than 0.45 or not substitutes 1 for yes and 0 for no.

18. The method according to claim 17, wherein the pre-confirmed reference value is determined by comparing the methylation levels of APC gene, COX2 gene, RASSF1A gene and miR-203 gene in one group of subjects known to non-live cancer with another group known to live cancer and obtaining a cutoff value according to the receiver operating characteristic (ROC) curve.

19. A kit for detecting methylation level of miR-203 gene, comprising:
a primer-pair including a sense primer and an antisense primer; and
a first probe and/or a second probe;
wherein the sense primer has a sequence of at least 85% sequence similarity to SEQ IDNO: 2, and the antisense primer has a sequence of at least 85% sequence similarity to SEQ IDNO: 3; and
wherein the first probe has a sequence of at least 85% sequence similarity to SEQ IDNO: 4, and the second probe has a sequence of at least 85% sequence similarity to SEQ IDNO: 5.

20. A kit for evaluating a risk of liver cancer in a subject and/or evaluating a prognosis of a subject afflicted with liver cancer, comprising:
a primer-pair and a probe for detecting methylation level of miR-203 gene;
a primer-pair and a probe for detecting methylation level of APC gene;
a primer-pair and a probe for detecting methylation level of COX2 gene; and
a primer-pair and a probe for detecting methylation level of RASSF1A gene.
